(19) United States
(12) Patent Application Publication
(10) Pub. No.: US 2024/0374183 A1
(43) Pub. Date: Nov. 14, 2024

(54) Separation Syringe and Processing Methods For Separation of Biological Fluids Into Fractions

(71) Applicant: NXTGEN Biomedical LLC, Chester Springs, PA (US)

(72) Inventors: Gino Bradica, Chester Springs, PA (US); Amit Deka, Media, PA (US); Christopher Steiner, Chester Springs, PA (US)

(73) Assignee: NXTGEN Biomedical LLC, Chester Springs, PA (US)

(21) Appl. No.: 18/782,849

(22) Filed: Jul. 24, 2024

Related U.S. Application Data
(63) Continuation-in-part of application No. 18/421,583, filed on Jan. 24, 2024.
(60) Provisional application No. 63/481,319, filed on Jan. 24, 2023.

(51) Int. Cl. A61B 5/15

(52) U.S. Cl. CPC A61B 5/150755

(57) ABSTRACT

A separation syringe assembly comprising a syringe barrel assembly and a plunger assembly, the syringe barrel assembly having an outer wall in the form of a hollow cylinder, and a cap provided at the distal end of the syringe barrel assembly; the plunger assembly providing a first plunger and a second plunger, the first plunger is a hollow barrel enclosed to form a container, the second plunger provides a cylindrical body with an open distal end and concentrically aligned outside of the first plunger. The separation syringe is configured to be rotated about the longitudinal axis and thereby separate the contents within the hollow cylinder of the syringe barrel assembly into at least two concentric annular layers.

### CROSS-REFERENCE TO RELATED APPLICATION

This application claims priority to and is a continuation-in-part of bypass U.S. patent application Ser. No. 18/421,583, filed on Jan. 24, 2024, and also to PCT Patent application No. PCT/US2024/012782, filed on Jan. 24, 2024, each of which claim the benefit of the filing date of Provisional Patent Application No. 63/481,319, filed on Jan. 24, 2023.

### TECHNICAL FIELD

The present invention pertains to devices for the separation of biologic fluids and biological connective tissues, and preparation of concentrated fluid fractions.

### BACKGROUND

With the increasing number of patients suffering from damaged or diseased organs and the shortage of organ donors, the need for methods to construct human tissues outside the body has arisen. Tissue engineering is a biomedical technology and methodology which combines the disciplines of both the materials and life sciences to replace a diseased or damaged tissue or organ with a living, functional engineered substitute. The so-called triad in tissue engineering encompasses three basic components identified as cell, scaffold, and signaling biomolecules.

Whatever the approach being used in tissue engineering, the critical issues to optimize any tissue engineering strategy toward producing a functionally equivalent tissue are the source of the cells, the substrate biomaterial to deliver the cells and particular signaling biomolecules where a regenerative process is required. Cells, and particularly stem cells (due to their unique properties) with signaling biomolecules are key requirements in a healing, repairing or regenerating damaged tissue. Briefly, certain types of cells have the ability to self-renew and commit to specific cell lineages in response to appropriate stimuli, providing excellent regenerative potential in healing tissue or likely leading to functionality of the engineered tissue. A major focus of tissue engineering, therefore, is controlling cell function in aiding the body to heal. There are many types of cells envisioned to be used in tissue engineering, based on their differentiation potential, can be divided into two categories: pluripotent stem cells and multipotent stem cells. Pluripotent stem cells include embryonic stem cells (ESCs) as well as induced pluripotent stem cells (iPSCs). Because ESCs are isolated from the inner cell mass of the blastocyst during embryological development, their use is still limited while more attention has been paid to adult stem cells, which are multipotent and have a large capacity to differentiate into a limited number of cell types. Adult stem cells can be found in many adult tissue types including bone marrow, peripheral blood, adipose tissues, nervous tissues, muscles, dermis, etc. For instance, mesenchymal stem cells (MSCs) which reside in the bone marrow can differentiate into bone (osteoblasts), muscle (myoblasts), fat (adipocytes) and cartilage (chondrocytes) cells, while neural stem cells (NSCs) either give rise to support cells in the nervous system of vertebrates (astrocytes and oligodendrocytes) or neurons. In vivo, differentiation and self-renewal of stem cells are dominated by signals from their surrounding microenvironment. This microenvironment or “niche” is composed of other cell types as well as numerous chemical, mechanical and topographical cues at micro- and nano-scales, which are believed to serve as signaling mechanisms to determine cell-specific recruitment, migration, proliferation, differentiation as well as the production of numerous proteins required for hierarchical tissue organization.

Biologic connective tissues are made up of cells and cell fragments suspended within and separated by non-living material (extracellular matrix). These tissues provide structural shape to the different organs while aiding to maintain their positions. Connective tissue is responsible for many different functions such as protecting against the invasions of pathogens by their phagocytic activity, storage of energy, and is involved in the transportation of water, nutrients, minerals, hormones, gases, wastes, and other substances within the body. Examples of connective include, but not limited to, cartilage, blood, bone, tendon, adipose, ligament, bone marrow, hemopoietic/lymphatic, placental and areolar tissues. There are three types of connective tissue: Fluid Connective Tissue, Fibrous Connective Tissue, and Skeletal Connective Tissue. These biologic connective tissues can be separated into their constituent parts, fragments, or fractions.

Therefore, it is important, when addressing a biological injury, to understand the tissue/tissues injured, the cells required to aid in the pathology of healing along with the biology of the cells that would be required to assist in the regenerative process. Understanding the translational science of these orchestrated events provides a blueprint of processes that should and should not be done during the separation of biological connective tissues into their constituent parts, fragments, or fractions to maintain viability, key niches or niche fragments/fractions.

Various systems exist to separate biological connective tissues, such as those used to create platelet rich plasma (PRP) or bone marrow concentration (BMC). Some use specialized test tubes, U.S. Pat. Nos. 7,179,391 and 7,520,402, that can include floats, tubing and/or gel materials of specific densities. Other systems use specialized double syringes, for example those found in U.S. Pat. Nos. 6,716,187 and 7,195,606. These test tubes and syringes must be centrifuged in a specialized large centrifuge for a specified time, typically 10-30 minutes, and then by delicate handling and extraction or decanting procedures produce the desired PRP/BMC. The consistency of these preparations can vary depending on the operator's skill level. Other systems, for example U.S. Pat. No. 6,982,038, contain specialized centrifuge chambers and complicated control systems to produce the PRP/BMC in about 30 minutes. All of these systems provide PRP/BMC of differing platelet concentrations depending on the method used. A major drawback to these methods is the need for an expensive piece of capital equipment which limits the utility to facilities that have the funds and space available. These methods also require considerable operator skills to complete the procedures necessary to obtain the PRP/BMC. Additionally, some require elaborate porting systems (U.S. Pat. Nos. 8,317,672 and 8,485,958) during the decanting procedure. This process creates unwanted vortices that can generate variability in desired output and require focused handling and extraction in order to capture the correct distribution of suspended biologic connective tissue parts.

The ability to produce PRP/BMC from a patient's own biological connective tissues at the point of care without the need for complex, expensive equipment and difficult procedures would facilitate the clinical utility of PRP/BMC. Therefore, the objects of this invention include among other things providing an apparatus and method for processing a patient's own biological connective tissues at the point of care in a short period of time that is self-contained, battery operated, small and or portable, inexpensive, easy to use, reproducible, able to separate a patient's biological connective tissues into their constituent parts, fragments or fractions to maintain viability, key niches, niche fragments/fractions, cellular populations, and disposable without the need for additional centrifugation equipment.

### SUMMARY

The teachings herein solve the problem associated with previously known separation systems, where it has been observed that blockages at discrete outlet ports can occur and impair the passage of fluid as it is exiting the separation chamber. In the embodiments taught herein, there is provided an annular exit opening, rather than discrete holes, thus, even if there was a blockage at a point within the annular opening, for example, due to clotting of red blood cells in a biologic tissue, the balance of the annular opening will still allow for passage of fluid. Thus, there is provided a more robust separation system. Additionally, many previously known systems require monitoring and are reliant upon user attention, judgement, and accuracy in reflexes or timing in determining when to actuate valves for ejection of fluid. The embodiment described herein will operate to separate the various fractions in an autonomous manner, and not be reliant upon user observation and discretion when separating the biologic fluids/tissues.

Further, in accordance with the teachings herein, in an embodiment there is provided a single use, sterile, self-contained, compact, easy to use centrifugal separation unit that provides for quick, reliable separation of fractions of biological connective tissues. For example, such a centrifugal separation unit as taught herein may be employed to provide platelet concentration from whole blood or bone marrow aspirate. The resultant Platelet Rich Plasma (PRP) and/or Bone Marrow Concentrate (BMC) can be immediately used for application to the patient, of is desired, stored under appropriate conditions for future use. The various embodiments taught herein are suitable for use in an office, operating room, emergency use, or military field hospitals.

In an embodiment, there is provided a self-contained separation syringe. Such a syringe could be utilized for processing various biological connective tissues. As used herein, the term “biological connective tissues” includes fluid connective tissues fluids containing various specialized cells circulating in a watery fluid containing salts, nutrients, and dissolved proteins. Many of the fluid connective tissues are rich in stem cells. Biological connective tissues contemplated for use in the various embodiments described herein include, as non-limiting examples, Blood, Marrow, lymph fluid, cerebrospinal fluid, Adipose, and Synovial Fluid. It is contemplated that the various embodiments of the self-contained separation syringe taught herein may be utilized to draw blood, and without requiring transfer to a new syringe or container, the same syringe the sample is drawn into may then be used to process the biological connective tissue by being centrifuged and allow separation of the fractions. The processing of the drawn sample, while remaining in the same container the sample is initially drawn into, avoids the need to transfer the collected sample between another container, thereby minimizing potential introduction of contaminants, and/or minimizing the possibility of rupturing red blood cells, and the associated initiation of a clotting cascade sequence, as might occur with extra transfers of the sample between chambers.

In an exemplary embodiment of the self-contained separation syringe, there is provided a concentric annular physical barrier system to separate the individual components during centrifugation, so that the fractions can be extracted. The various embodiments of the biological connective tissue separation device taught herein serve to separate a discrete sample of biological fluids or tissues into component fractions by specific gravity, then physically isolate each fraction in a reliable manner and also maintain the cellular viability within each fraction. It is contemplated that any one or more of the isolated fractions processed according to the embodiments described herein may beneficially be applied to a treatment site of a patient, or preserved for future use as will be familiar to those of skill in the art.

In an embodiment, a separation syringe assembly is provided. The separation syringe may have a syringe barrel assembly and a plunger assembly configured to travel relative to the syringe barrel, the syringe barrel assembly may have a distal end and a proximal end with a longitudinal axis extending therebetween, the syringe barrel assembly may have an outer wall in the form of a hollow cylinder, an inner wall in the form of a hollow cylinder capable of being axially translated and arranged concentrically within the outer wall, a drain actuator may be provided secured to the proximal end of the inner wall, and a cap may be provided at the distal end of the syringe barrel assembly; the plunger assembly may have a proximal end and a distal end and aligned with the longitudinal axis, the plunger assembly may provide a first plunger and a second plunger, the first plunger may be a generally cylindrical hollow barrel having an entrance port near the distal end of the first plunger and enclosed on the proximal end, the second plunger may provide a cylindrical body with an open distal end and concentrically aligned outside of the first plunger.

In an embodiment, the syringe barrel assembly may have an inner and outer chamber, the inner chamber may be provided within the inner wall, and an outer chamber may be provided within the outer wall and exterior to the inner wall. A distal seal may be provided near the distal end of the outer wall, adjacent to an interior aspect of the cap, and may be capable of selectively sealing against the axially translatable cylindrical inner wall. In an embodiment, the inner wall may be reciprocated between a first position and a second position, wherein the first position is characterized by urging the inner wall in a distal direction to be sealed against the distal seal; and wherein the second position is characterized by urging the inner wall in proximal direction in an amount that creates an annular passage adjacent to the distal seal, the annular passage establishing fluid communication between the inner chamber and the outer chamber. The inner wall may further comprise a vent opening that can selectively be opened to create fluid communication between the outer and inner chambers, and the drain actuator may be moved by an actuation mechanism acting upon the syringe barrel assembly, causing the axial translation of the inner wall. In an embodiment, the inner wall may further comprise a ridge portion protruding near the distal end of the inner wall and arranged as a generally annular ring extending entirely around an exterior surface of the inner wall and extending toward an inner surface the outer wall. Such a ridge portion may create a fluid channel between the ridge portion and the inner surface of the outer wall. In an embodiment, at least a portion of each of the outer wall, inner wall and ridge portion are optically transparent. The first plunger may have a one-way valve positioned adjacent to the entrance port. The first plunger may have an inverse conical face at the distal end of the first plunger, with the entrance port provided at the peak of the inverse conical face.

In an embodiment, the cap may be provided with a conical feature that is centrally aligned on an inside face of the cap, the conical feature capable of engaging with the inverse conical face of the first plunger. The cap may further be provided with a first access passage in fluid communication with the inner chamber and offset from the longitudinal axis.

In an embodiment, the first access passage features a luer fitting, and can be closed with a cap. The cap may be provided with a second access passage that is aligned with the longitudinal axis, the second access passage providing a pierceable septum. The cap may be provided with a counterbalance offset from the longitudinal axis in the same amount as the first access passage and in an opposite direction, the counterbalance having a weight that is the same as the luer fitting and cap.

In an embodiment, the syringe assembly may be rotated about the longitudinal axis. In an embodiment, the first plunger and second plunger are each capable of independent axial translation along the longitudinal axis. In an embodiment, the syringe barrel may further comprise at least one support element configured to stabilize the syringe barrel and limit the lateral movement of the inner wall relative to the outer wall. The support element may be a rod arranged along a central axis of the syringe assembly; or a plurality of stabilizing tabs positioned between the outer wall and the inner wall. In an embodiment, a distal end of the axially translatable cylindrical inner wall may have a tapered surface to selectively conform against the distal seal when the cylindrical inner wall is urged distally.

In an embodiment, there is provided a separation syringe assembly having a syringe barrel assembly and a plunger assembly configured to travel relative to the syringe barrel, the syringe barrel assembly having a distal end and a proximal end with a longitudinal axis extending therebetween, the syringe barrel assembly having an outer wall in the form of a hollow cylinder, and a cap provided at the distal end of the syringe barrel assembly. The plunger assembly may have a proximal end and a distal end and aligned with the longitudinal axis, the plunger assembly providing a first plunger and a second plunger, the first plunger may be a generally cylindrical hollow barrel having an entrance port positioned about the longitudinal axis and near the distal end of the first plunger and enclosed on the proximal end to form a container therein, the distal end of the first plunger may be complementary in shape to a corresponding face of the cap of the syringe barrel assembly, the second plunger may provide a cylindrical body with an open distal end and concentrically aligned outside of the first plunger. In an embodiment, the separation syringe is configured to be rotated about the longitudinal axis and thereby separate the contents within the hollow cylinder of the syringe barrel assembly into at least two concentric annular layers.

In an embodiment, the separation syringe assembly may provide an entrance port of the first plunger having a one-way valve positioned adjacent to the entrance port. The separation syringe assembly may have the first plunger featuring an inverse conical face at the distal end of the first plunger, with the entrance port provided at the peak of the inverse conical face. The separation syringe assembly may provide the cap having a conical feature that is centrally aligned on an inside face of the cap, the conical feature capable of engaging with the inverse conical face of the first plunger. In an embodiment, the cap may be further provided with a first access passage in fluid communication with the inner chamber, and offset from the longitudinal axis. In an embodiment, the first access passage may feature a luer fitting, and can be closed with a cap. The cap may be further be provided with a second access passage that is aligned with the longitudinal axis, the second access passage providing a pierceable septum. The cap may be provided with a counterbalance that is offset from the longitudinal axis in the same amount as the first access passage and in an opposite direction, the counterbalance having a weight that is the same as the luer fitting and cap.

In an embodiment, the separation syringe assembly may provide the first plunger and second plunger being capable of independent axial translation along the longitudinal axis. The first plunger may further comprise at least one support element configured to stabilize the first plunger and limit the lateral movement of the first plunger relative to the syringe barrel. The at least one support element may be a plurality of stabilizing tabs positioned between the outer wall and the inner wall.

### BRIEF DESCRIPTION OF THE DRAWINGS

The invention will be explained in greater detail in the following with reference to embodiments, referring to the appended drawings, in which:

FIGS. 1A and 1B depict a perspective view of the operation of a separation syringe provided to draw a sample from a donor into the syringe, according to an exemplary embodiment of the invention;

FIG. 2 depicts a cross-section view of the syringe of FIG. 1 containing a biologic fluid tissue within an inner chamber, according to an exemplary embodiment of the invention;

FIGS. 3A and 3B depict a representative view of the syringe of FIG. 2, positioned in a centrifuge system for rotating the syringe about the longitudinal axis and having separated the biologic fluid tissue into three distinct fractions within the inner chamber, according to an exemplary embodiment of the invention;

FIG. 4 through 4D depicts an enlarged view of the distal portion of the syringe of FIG. 3B, according to an exemplary embodiment of the invention, and an exemplary processing method for isolating the first fraction within an outer chamber;

FIG. 5A through 5C depicts the cross-section view of the syringe of FIG. 4D, with the 3^{rd} and 2^{nd} fraction remaining within the inner chamber, and the 1^{st} fraction within the outer chamber, and an exemplary processing method for isolating the second and third fractions, according to an exemplary embodiment of the invention;

FIG. 6 depicts the syringe of FIG. 5C, with the separation syringe removed from the centrifuge unit, and the third fraction isolated within the inner chamber, according to an exemplary embodiment of the invention;

FIG. 7 is a partial exploded view of the components of the separation syringe of FIG. 2, according to an exemplary embodiment of the invention;

FIGS. 8A and 8B depicts an alternative seal assembly for the separation syringe providing an overcomeable valve arrangement showing the selective flow of the first fraction through the annular passage for isolating the first fraction, according to an exemplary embodiment of the invention;

FIG. 9 depicts a cross section view of an alternative distal tip design utilizing a plurality of annular channels provided in a funnel tip for isolating fractions from a separation centrifuge, according to an exemplary embodiment of the invention;

FIG. 10 depicts a cross-section view of an alternative embodiment of a separation syringe, having a support element;

FIG. 11 depicts an enlarged view of the syringe provided with at least one support element in the form of a support tab according to an exemplary embodiment of the invention; and

FIG. 12 depicts a cross section view of an alternative embodiment of the separation syringe for separating a biologic fluid into two fractions.

### DETAILED DESCRIPTION

In accordance with the invention, a single use, sterile, self-contained, compact, easy to use centrifugal separation syringe 10 that provides for quick, reliable platelet concentration from whole blood. The resultant PRP or platelet concentrate can be immediately used for application to the patient. The unit is suitable for office, operating room, emergency use, or military field hospital use.

The disposable separation syringe 10 may be provided with a motorized separation centrifuge unit (not shown) featuring a motor with a drive axis. In an embodiment, the centrifuge unit has a drive axis that is coaxial with the central or longitudinal axis 6 of the separation syringe 10. In another embodiment, the centrifuge unit has a motor with a drive axis that is generally parallel to, but not necessarily coaxial to the longitudinal axis 6 of the separation syringe. It is contemplated that other transmission alternatives for rotating the separation syringe are possible, and such variants may be capable of rotating the separation syringe around the longitudinal axis. The motor of the centrifuge unit may have the capacity to rotate the disposable separation syringe at speeds in the range 5,000 to 30,000 RPM for several minutes. Power can be supplied to the motor through a battery or other power pack. The power can be connected through a switch and even small dry cell batteries will have sufficient capacity to complete the separation process. Alternatively, the separation syringe 10 can be rotated by non-electrical means such as an air driven turbine or spring drive. It could also include a magnetic or mechanical coupling to an external drive motor, or any source of energy that may be available at the Surgical site for example in the Surgical Suite or on location during a trauma procedure, such as at a “MASH” compound. The disposable unit may be provided independent of, or along with the centrifuge unit as a kit, along with instructions for use. The separation syringe 10 includes an access port for introducing a sample therein, such as a luer fitting or other fitting known in the art. The separation syringe is also provided with a luer fitting to which a needle, or another syringe or container may be mounted, for ejecting the isolated fraction from the inner chamber (e.g., the PRP). The separation syringe also provides a pierceable septum, through which a syringe needle can be inserted to retrieve all or some of the isolated fraction from the interior of the PPP plunger (e.g., the platelet poor plasma).

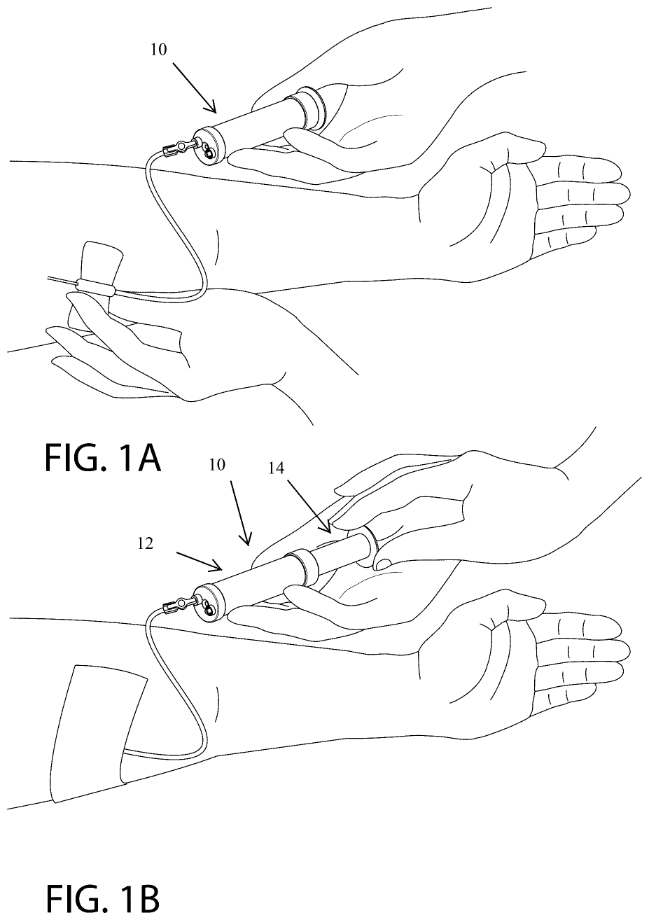

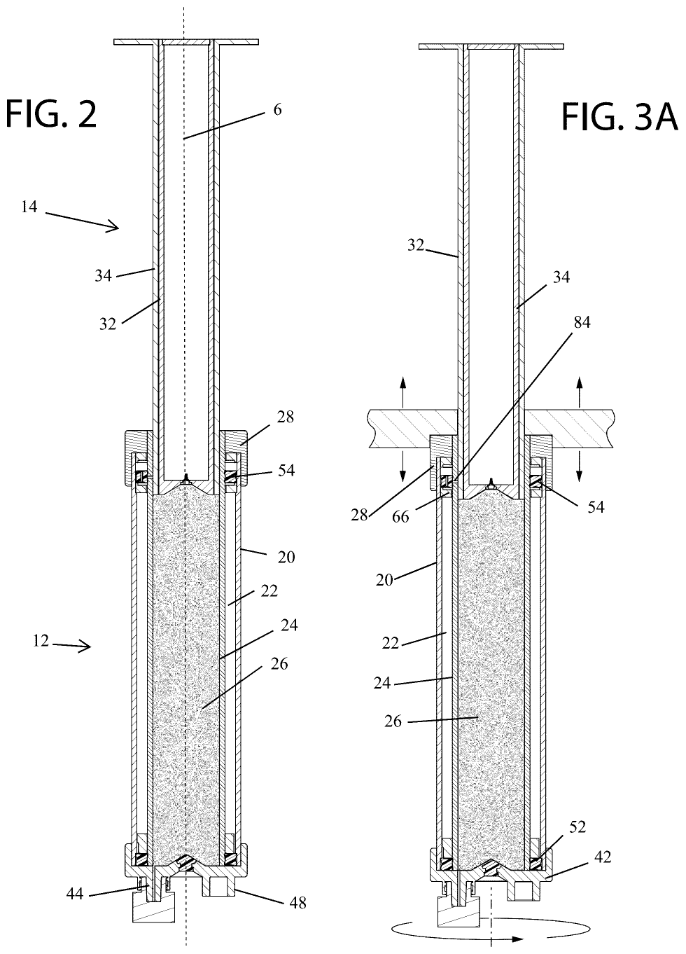

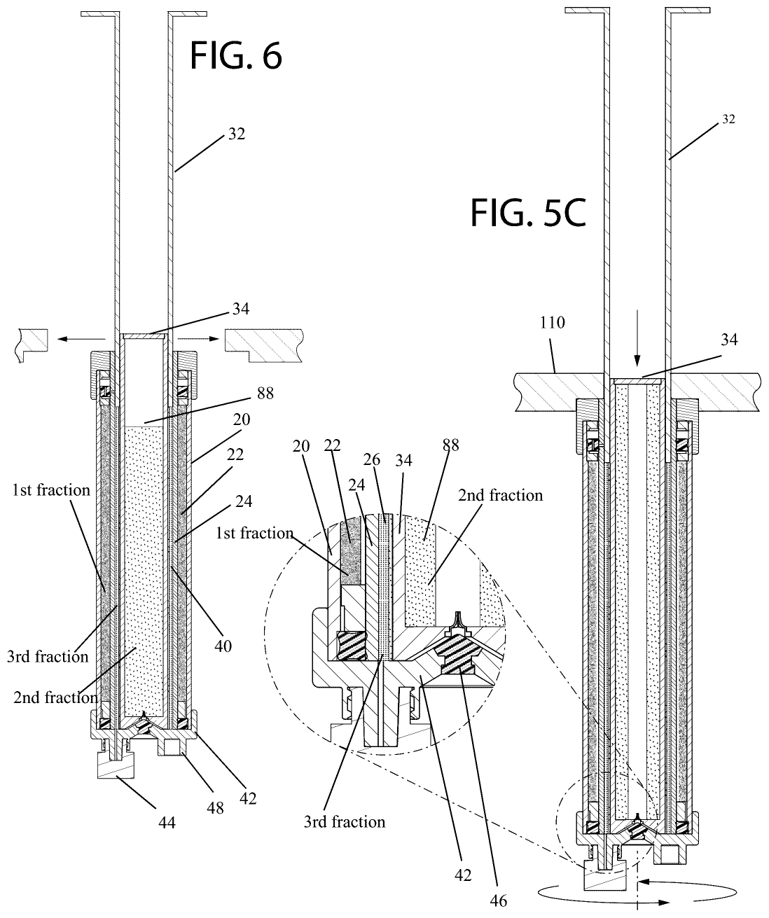

In an embodiment, and with reference to FIGS. 1A-8B and 10, the separation syringe 10 has a syringe body 12 featuring concentric barrels, some of which are capable of being axially translated to some degree. When the separation syringe is provided with the plunger 14 assembly fully advanced into the syringe body 12, as depicted in FIG. 1A, the concentric components include, as can be seen with reference to FIG. 2, when viewed from outside the syringe and examined in a direction towards the central longitudinal axis 6, there is provided an outer chamber wall 20, an outer chamber 22, the RBC/PRP wall 24 and the inner chamber 26. The axially movable components of the plunger assembly 14 are depicted having been retracted out from the syringe body 12 in FIG. 2, and include, the PRP plunger 32, and the hollow PPP plunger 34. An exploded view of the components of the syringe assembly 10 are depicted in FIG. 7 and assembled views in various stages of operation in FIGS. 1-6. As depicted, the syringe assembly 10 is a generally cylindrical form, and having a distal end cap 42 at the distal end, with the movable components (RBC slider drain actuator 28, RBC/PRP wall 24, and plunger assembly 14, being capable of being repositioned relative to the distal end cap 42 and the outer chamber wall 20, in order to selectively open a passageway, or to physically separate and isolate the fractions of the sample that have first been arranged into concentric annular layers by centrifugation. In an alternative embodiment, there may be provided an overcomeable valve that has a cracking pressure for opening a passageway as the separation syringe 10 is rotated, rather than the axially translatable RBC drain actuator components.

According to an exemplary embodiment, the separation syringe as utilized for processing a discrete sample of biological connective tissue provides a syringe body assembly and a plunger assembly. The syringe body assembly comprises the following major parts: Outer Chamber Wall 20; RBC/PRP Wall (RBC Separator) 24 of the RBC Actuator assembly 30; RBC Slider Drain actuator 28 of the RBC Actuator assembly 30; and a Distal End Cap 42. The plunger assembly 14 having a PRP/PPP Wall (PPP Separator Plunger) 34; and a PRP Plunger 32 having a flap valve 36.

A partially exploded view of an exemplary separation syringe 10 can be seen with reference to FIG. 7. The Distal End Cap 42 is circular in nature and is a physical element that allows for structural containment of the Outer Chamber 22, RBC/PRP Wall 24 and Plunger Assembly System 14 (PPP plunger 34 and PRP Plunger 32). The Distal End Cap 42 not only structurally contains the distal end of the separation syringe 10 but allows for the distinct placement of the luer fitting 44, a septum 46 and allows for the annular orifice pathway. The luer fitting 44, a threaded element, allows for drawing biological tissues into the inner chamber 26 of the separation syringe 10 and the removal of the separated PRP biological tissues. The septum 46, a membrane similar to those of medication vials, is placed in a portion of the End Cap 42 and allows for the removal of the separated biological tissue within the PPP separator plunger 34. Finally, the annular orifice pathway is created when the RBC slider drain actuator 28 including the RBC/PRP Wall 24 is separated from the Distal End Cap 42, during the linear motion of the RBC/PRP Wall Complex 24 allowing for an annular passageway to be temporarily created between the RBC/PRP Wall 24 and Distal End Cap 42 for the separated biological tissues to be removed from the inner chamber 26 of the syringe 10 to the outer chamber 22 (between the Outer Wall 20 and the RBC/PRP Wall 24).

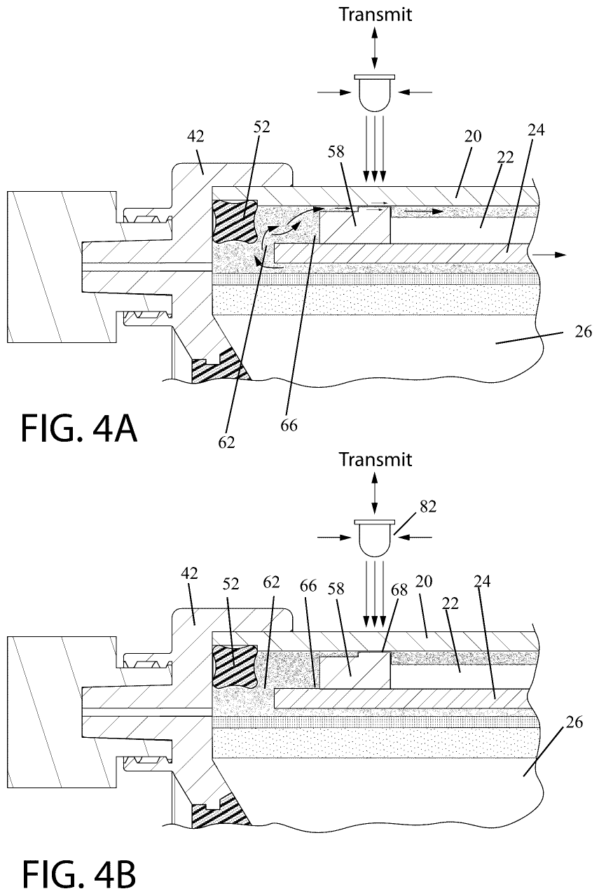

The outer chamber wall 20 is a hollow cylindrical barrel form and provides a physical structure for the device and a portion of the maintenance of the first biological fluid to be contained. The outer chamber wall 20, as shown may be a generally cylindrical barrel, that forms the exterior barrel of the separation syringe 10. The outer chamber wall 20 may be enclosed by receiving the distal cap 42 on the distal end of the outer chamber wall 20, and the RBC slider drain actuator 28 on the proximal end. Additional components may be received within the interior of the outer chamber wall 20, as will be discussed. The outer chamber wall 20 may be an optically transparent or translucent material, such that a light detection system 82 for monitoring the movement of the contents within the outer chamber 22 may be monitored, as will be discussed. The distal end of the outer chamber wall 20, along the interior surface may be provided with a seal retention portion 64, such as an inward facing recess, as shown in detail in FIG. 4, that can receive a portion of the distal seal 52 therein.

The RBC/PRP Wall 24 (RBC Separator) provides a physical structure for the separation of the first biological fluid (between itself and the inside surface of the outer chamber wall 20). The RBC/PRP wall 24 is a generally cylindrical barrel that concentrically fits within the dimensions of the outer chamber wall 20. At a distal end of the RBC/PRP wall 24, there may be provided a distal seal 52, such as an O-ring, quattro seal or any other type of seal needed to create a seal and allow for creating an impermeable passage at the interface of the RBC/PRP wall 24 when advanced distally, but is in a sliding relationship with the RBC/PRP wall 24 such that when the RBC/PRP wall is retracted proximally by a small amount, an annular opening 62 is created to allow controlled fluid past the distal seal 52. Thus, the distal seal 52 is in sliding engagement with the exterior surface (away from the longitudinal axis) of the RBC/PRP Wall 24 and will reside at the junction of the outer chamber wall 20 with the distal cap 42. The RBC/PRP wall 24 may be optically transparent to allow for the use of a light detector system 82 for monitoring the movement of the contents within separation syringe 10.

The light detector system 82 may be any suitable optical detector, such as an LED based light and sensor system, to act as a feedback mechanism for opening and closing the annular opening 62, based on the detected characteristic of the fluid passing through the sensor beam, such as the optical density or a ratio of wavelengths of absorbed spectra detected, as measured within the path of the beam.

The RBC slider drain actuator 28 may interact with any suitable mechanism for causing the lateral translation of the RBC/PRP separator wall 24 within the syringe body 12, for example, using a yoke and bearing assembly as will be familiar to those of skill in the art. Alternatively, a fork or plurality of engagement features of the lateral translation mechanism may engage with features or grooves provided on the exterior aspects of the RBC slider drain actuator to cause the lateral translation, relative to the outer chamber wall 20 of the RBC slider drain that forms the proximal end cap of the syringe body 12.

At the proximal end of the RBC/PRP wall 24 may be provided a proximal gasket 54 and locator ring 56 that are mechanically engaged with RBC slider drain actuator 28. The proximal gasket 54 is fitted into a groove within the locator ring 56 and provides a seal against the interior surface of the outer chamber wall 20. The RBC/PRP wall 24 may be caused to translate along the longitudinal axis 6, thereby creating an annular passage 62 as shown in FIGS. 4A-4C, which can be selectively opened at a point near the distal end cap 42, and/or near the distal end of the RBC/PRP wall 24. The annular passage 62 may be selectively opened, using the RBC Actuator 28, to thereby allow for the passage of the first fraction of the biological fluid. The extent that the RBC actuator 28 is actuated will dictate how far the RBC/PRP wall 24 is moved away from the distal seal 52. Near the distal end of the RBC/PRP wall 24, but slightly set away from by approximately the thickness dimension of the seal 52, there is provided a seal recess 66 of the RBC/PRP wall 24, into which the distal seal 52 would conform when the RBC actuator 28 is closed. As shown in the cross-sectional view of the device, specifically with reference to FIG. 4, the RBC/PRP Wall 24 may be provided with a protruding annular ridge portion 58. This annular ridge portion serves to restrict the flow through the annular passage 62 when the passage is open for fluid flow. The ridge portion 58 serves to direct the flow of the first biological fluid from the annular passage 62 and pass through the light beam of a sensor system 82. As depicted, the ridge portion 58 may be a stepwise reduction in the flow channel, characterized by providing different levels of reduction in incremental amounts along the passage, or alternatively, the ridge may provide a smooth reduction, by presenting an arced or curved surface extending from the distal end of the ridge, from the interface of the ridge with the exterior surface of the RBC/PRP wall 24, and when viewed in a proximal direction, thereby creating a narrowing of the annular passage. This narrowed annular passage serves to provide a narrowing 68 of the fluid passage between the widest extent of the annular ridge portion 58 (extending away from the longitudinal axis), and at a position facing the interior surface of the outer chamber wall 20. In the embodiment where the ridge 58 provides a smooth reduction, the passage of the first biological fluid may proceed in a manner that minimizes disruption to the flow of the first biological fluid, such as by largely preserving laminar flow of the first biologic fluid. Alternatively, where the narrowing of the passage is step-wise, as shown in 4a, with the flow of fluid indicated by the arrows, may be somewhat disturbed passing over the annular ridge portion 58, and no longer flow in a laminar manner, where the non-laminar flow may be useful in controlling the rate of passage of the first biological fluid fraction through the annular passage 62, but the extent of the disturbance for the non-laminar flow should not be so great as to initiate a clotting cascade due to shear of the cells in the fraction. Additionally, it is recognized that even if clotting cascade is initiated, the clotting effect would occur within the outer chamber, as the flow would have taken the affect portion past the annular narrow before being affected. It is contemplated that the dimensions of the annular ridge 58 may be of different design or dimensions, in order to be suitable for use with a variety of biological fluids, for example, those having different viscosities or fluid flow characteristics. The annular ridge 58 may be varied, for example, by adjusting any one or more of the dimensions of the ridge, the shape of the ridge, and the resulting thickness of the narrowed portion 68 of the annular passage through the light beam pathway. It is contemplated that varying the surface features of the annular ridge portion 58 (e.g., smooth gradation or the stepwise profile, as shown) may controllably improve the efficiency of separation and the performance of the separation device 10. With reference to FIG. 4A, the annular ridge portion 58 ensures that the flow rate through the annular opening 62 is reduced within the narrow 68 to the point where the flow rate of the first biological fluid fraction is in the desired range. Control of the flow rate through the annular passage 62 and narrow 68 may provide benefits in achieving one or more of the following: a desired cycle time for separation and isolation of fractions from a biologic fluid mixture through the operation of the device; may serve to minimize damage to the cells of the collected, isolated fractions; may maximize the efficiency of the separation device, by ensuring the laminar separation between the fractions is maintained through processing; and may further ensure that the flow of the different fractions of the sample within the syringe 10 will not disrupt the interfaces created at the boundaries between the different layers, such as those remaining within the inner chamber 26.

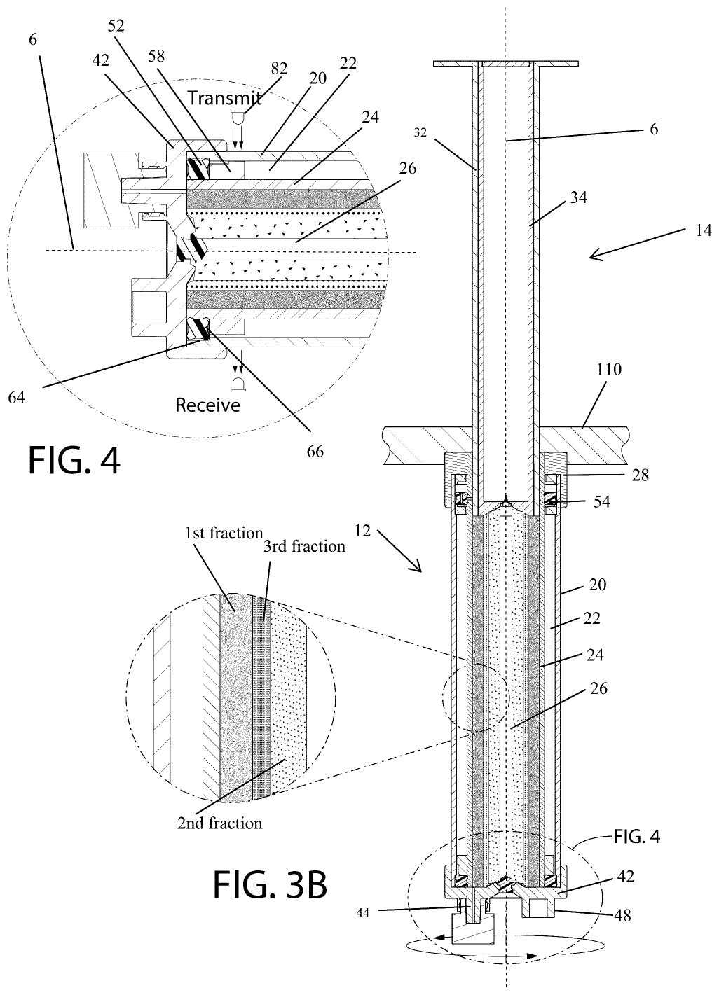

The RBC Actuator/RBC Slider Drain 28 encloses the proximal end of the Outer Chamber (wall) 20 and is connected to the distal end of the RBC/PRP Wall (RBC Separator) 24. The RBC Actuator 28 comprises a circular, partially open cap that is physically attached to the RBC/PRP Wall 24. Together, the RBC actuator 28, and the RBC/PRP Wall 24 create a movable RBC drain assembly 30 which can slide relative to the Outer Chamber 22. When the separation syringe 10 is placed within the centrifugation device, which may be any suitable mechanism to allow longitudinal rotation of the separation syringe about its longitudinal axis 6, a longitudinal mechanical displacement drive 110 connects to the RBC Actuator 28 in order to selectively control the sliding movement of the movable assembly 30. As the separation syringe system is rotating about the longitudinal axis and the separation of the biological tissue occurs, the longitudinal mechanical displacement drive moves the RBC Actuator/RBC/PRP Wall complex 30 proximally along the longitudinal axis 6, relative to the outer chamber 22, thereby creating an annular opening 62 at the proximal end between the RBC/PRP Wall 24 and the Distal End Cap 42. This can be seen with reference to FIG. 4, where the RBC actuator 28 is in a closed position, and comparing to FIG. 4B, where the RBC actuator 28 is in an open position. Simultaneously as this motion occurs, a venting system is opened at the proximal end between the inner chamber 26 and the outer chamber 22, as the proximal gasket 54 within the locator ring 56 is caused to slide proximally, revealing a vent hole 84, which can be seen with reference to FIG. 3A, positioned extending through the thickness of the RBC/PRP separator wall 24, and shown with the opening currently sealed by the positioning of the proximal gasket 54. Once the vent opening 84 is no longer sealed, due to the proximal displacement of the scaling proximal gasket as the RBC actuator urges the movement of the proximal seal in a proximal direction, the now open vent 84 allows for the equalization of pressures within the outer chamber 22 and the inner chamber 26, as a first portion of the separated biological tissue is caused to be decanted past the annular ridge portion 58, and into the outer chamber 22 area between the Outer Chamber (Wall) 20 and the RBC/PRP Wall 24, while the air within the outer chamber 22 is vented via the small port 84 of the venting system back into the inner chamber 26 and allowing for Biological Tissue flow, as depicted in FIG. 4A. It is contemplated that such a vent hole 84 may be capable of being of any suitable size, or shape for passing a fluid gas therethrough. It is further contemplated that restricting the flow of fluid gas through the vent opening 84 may allow the vent system to serve as a limit to the rate of transfer of the first fluid fraction through the annular passage 62. A vent opening 84 may be dimensioned to limit the rate of decantation of the first fraction, in an indirect manner, as the dimensions of the vent hole 84 may limit the rate of pressure equalization between the outer and inner chambers 26, 22, thereby slowing the rate of decantation of the first fluid fraction. By controlling the rate of equalization with the vent hole 84, the rate of decantation of the first fluid is not being slowed by direct physical restrictions acting directly on the fluid fraction, but rather via pressure differentials, thus avoids potentially creating issues that might occur due to shear, for example, as might be likely where a fluid containing cells is passed quickly through a restrictive opening, where the physical dimensions of the opening might damage the cells as they are passing rapidly through the narrow restricted opening. The present invention seeks to limit the transfer rate between chambers through a variety of methods, and therefore need not rely entirely upon the sizing of the passage past the ridge portion 58 to achieve the desired flow rate. Rather, the narrow portion of the passage past the ridge portion 58 is to provide a uniform sample area for the sensor system to detect characteristic properties of the fluid passing through the detector beam and need not be so narrow as to damage the cells passing through.

During the flow, the light detector system 82 monitors one or more characteristics of the material passing through the narrow within the sensor beam, such as the optical density recorded in the beam, or comparing a ratio of absorbed spectral frequencies, as depicted in FIG. 4B. The cells for each fraction may comprise a gradient within their range of targeted specific gravity, thus, there may be some overlap between the fractions with regard to the specific gravity. With reference to FIG. 4C, once the light detector system 82 provides the feedback mechanism that the interface between the first fraction, and the second fraction is detected, or alternatively, determines that a predetermined quantity of the first biological tissue is captured within the outer chamber 22, the light detector system 82 provides a feedback signal to stimulate the longitudinal mechanical displacement drive 110 to move the RBC Actuator/RBC/PRP Wall complex 30 in a distal direction, and it is returned to the original closed position, depicted in FIG. 4D. It is contemplated that there may be a desire to halt the flow of the first fraction into the outer chamber at a point where only majority portion of the fraction with the highest specific gravity has passed. For example, where the sample is whole blood, by monitoring the detected values from the sensor system, the values could be compared to a known standard library for the values (e.g., detecting less than 10% of the nominal value for RBC; detecting less than 8% of the nominal value for RBC; detecting less than 5% of the nominal value for RBC; detecting less than 2% of the nominal value for RBC) representative of different percentages of RBC to other constituents, such as PRP. This allows user adjustment of how stringent the separation fraction will be in excluding the presence of red blood cells. In an exemplary instance where there is more emphasis on preserving all of the PRP within the inner chamber, a less stringent standard (e.g., at a detected value that is less than 20% of the nominal RBC value) would be applied, which would close the fluid flow early, leaving more red blood cells within the inner chamber, but to a statistical near certainty capturing all of the PRP in the inner chamber. Conversely, a more stringent standard (e.g. at a detected value of 1% of the nominal RBC value) may be applied there is more emphasis on excluding substantially all of the RBC from the sample preserved in the inner chamber, but at the expense of the final volume of the PRP remaining; in such an instance a late closure of the annular passage (relative to the positioning of the interface) would likely result in some of the PRP cells in the outer chamber, but exclude far more of the red blood cells. Once the desired value range is detected, the microprocessor may trigger the closing of the annular passage, in order to isolate the desired fraction makeup in the outer chamber and leave the remainder within the inner chamber for further processing.

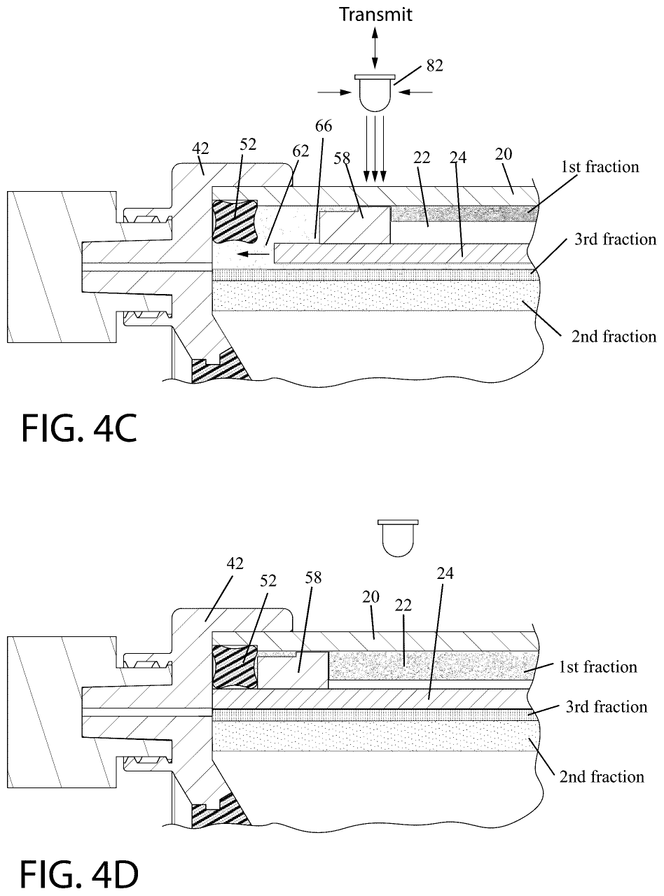

The Plunger Assembly 14 is made of the PRP Plunger 32 and the PPP Plunger 34 which incorporates a flap valve 36. The plunger assembly 14 is configured to allow for the PPP Plunger 34 to operate together with, or separate from, the PRP Plunger 32. The Plunger Assembly 14 components function together during the drawing of biological tissues as both the PPP Plunger 34 and PRP Plunger 32 are drawn back to fill the inner chamber 26, within the interior of the RBC/PRP wall 24, with biological tissue. The two plunger components of the plunger assembly 14 may then function independently post-centrifugation. Initially, the PPP Plunger 34, provided as a hollow cylindrical chamber, having a annular tapered distal end, and an inversely conical entrance port 38 providing the flap valve 36 at the peak of the conical entrance, as can be seen with reference to FIG. 7, near the distal end of the PPP plunger 34. The Flap Valve 36 is positioned at a PPP entrance port 38, positioned at the peak of the inversely conical entrance of the PPP separator plunger. Once the biologic tissue is separated through centrifugation, and the first fraction captured in the outermost chamber, the PPP separator plunger 34 may be advanced in order to isolate the second fluid fraction, such as the plasma portion, from the third fluid fraction, such as the PRP. When capturing the second fluid fraction, the PPP Separator Plunger 34 is to be driven longitudinally into the interior chamber 26 of the separation syringe 10, concentrically advanced within the interior of the RBC/PRP wall 24 component of the RBC drain assembly 30. The movement of the PPP plunger 34 may be manually operated, or by an automated or semi-automated manner, such as through a linear drive mechanism within the centrifuge device that can controllably and smoothly advance the PPP plunger 34 into the syringe body 12. Such a linear drive mechanism (not shown) may rely on an electronic motor or may be advanced by a transmission actuated with the rotation of the centrifuge unit as it axially spins the separation syringe. This linear motion allows the Plasma portion of the Biological Tissue to be urged through the flap valve 36, and into the hollow portion or PPP containment area 88 within the interior of the PPP Separation Plunger. The inverse conical shape of the distal end of the PPP plunger 34 extends from the annular leading edge of the plunger and directs the second biologic fluid toward the PPP entrance port 38. The annular leading edge of the PPP plunger will generally be aligned just to the interior (toward the central longitudinal axis 6 of the separation syringe 10) of the interface between the second biologic fluid fraction (the Plasma) and the third biologic fluid fraction (the PRP). The Flap Valve 36 is used to regulate the flow of the innermost layer of biological fluid, as it may pass unrestricted into the PPP containment chamber 88, as the flap valve is a one-way valve, but the flap valve 36 prevents fluid flow in the reverse direction, to ensure that the second fraction remains contained within the PPP plunger 34. The flap valve 36 may be a duckbill valve as known in the art. The transfer of Biological Tissue continues until the PPP Separator Plunger 34 is driven linearly to come in full contact with the Distal End Cap 42 of the Separation Syringe 10. As can be seen with reference to FIGS. 5B and 5C, the inner face of the distal end cap 42 is provided with a protruding conical aspect, with the peak aligned with the central axis 6. The PPP plunger 34, with the inversely conical distal end, will matingly engage with the central peak of the cap 42, leaving minimal gap remaining once the PPP plunger 34 is urged fully in a distal direction, thereby forcing substantially all of the second fraction through the PPP entrance port 38 into the PPP plunger chamber 88. As will be discussed, the PRP plunger 32 may be actuated separately from the PPP separator plunger 34, where the PRP plunger may be advanced and eject the third fluid fraction, such as the PRP, from the interior of the RBC/PRP wall 24, and out from a luer fitting 44 provided on the distal end cap.

Once the centrifugation and separation of the fractions of the biologic fluid mixture is completed, the Separation Syringe 10 can be removed from the centrifuge device with a Separation Syringe that has isolated the basic elements of a Biological Tissue into three distinct containment vessels: 1: the Outer Chamber 22, located between the Outer Wall 20 and RBC/PRP Wall 24; 2) the PPP Chamber 88 corresponding to the area maintained within the interior of the PPP Separator Plunger 34; and 3) the PRP Chamber 40 corresponding to the area maintained between the RBC/PRP Wall 24 and the PPP Separator Chamber 34. Of the three fractions, only the middle fraction remains within what was originally the inner chamber, whose dimensions were reduced by the introduction of the PPP plunger.

The PRP Plunger 32 is an annular structure used to evacuate the remaining biological tissue, as the cylindrical body of the PRP plunger 32 is slidingly advanced through the RBC slider drain actuator 28 and is further advance concentrically around the PPP plunger 34 and concentrically within the RBC/PRP wall 24. The PRP Biological Tissue can be directly dispensed out the luer fitting 44, and may be then administered to a treatment site of a patient, or transferred to another container, such as a syringe, cup, device, biological sponge, biomaterial, etc. The linear movement of the PRP Plunger 34 will eject the PRP Biological Tissue via the Luer Fitting 44 of the Distal End Cap 42. As the luer fitting 44 is offset from the center axis of the syringe in order to provide the luer fitting 44 in alignment with the PRP plunger; there may be a need to provide a counterbalance to the weight attributable to the capped luer fitting, and positioned on the cap in a mirrored positioned across the longitudinal axis, with each of the luer fitting 44, the center of the cap, and the positioning of the counterbalance 48 being positioned on a line that is a diameter of the cap 42 portion. This counterbalance 48 can be seen with reference to FIGS. 2 and 7, where the counterbalance may be a molded portion of the distal cap 42, with a weight that approximates the capped luer fitting 44, as it is anticipated that the luer will be capped during centrifugation of the separation syringe 10.

Additionally, if the PPP Biological Tissue is determined to be needed, a separate syringe with a needle can be used by allowing positioning the separate syringe with the needle caused to puncture the Septum 46 of the Distal End Cap 42 and pass through the Flap Valve 36 of the PPP Plunger 34 into the PPP Separator Chamber 88. The necessary amount of PPP Biological Tissue can be extracted from the PPP Separator Chamber 88. Once completed, the Separation Syringe can be disposed of following standard procedures for disposing of biological waste.

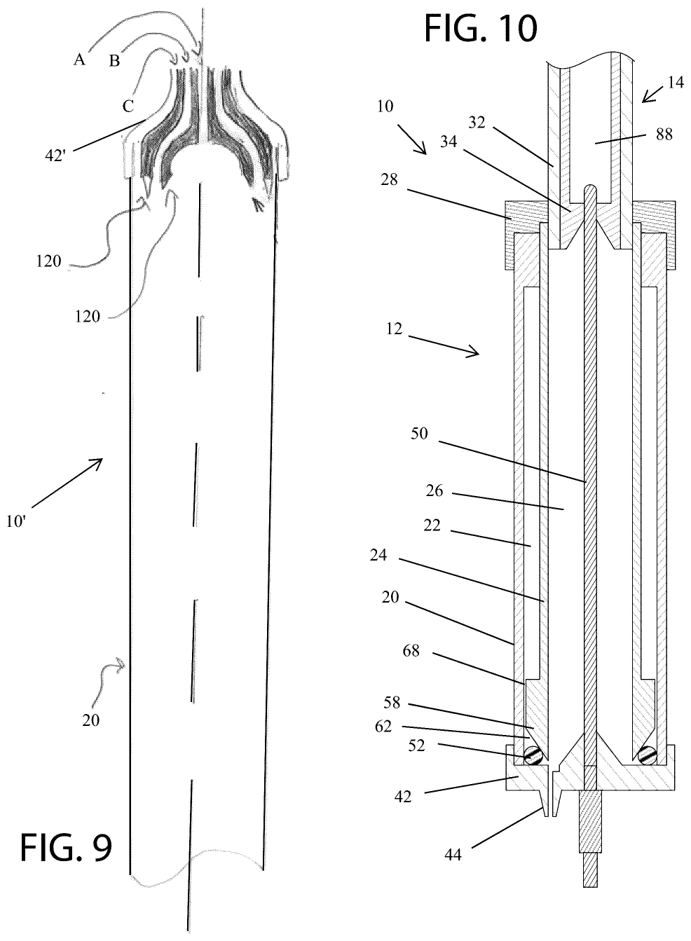

With reference specifically to FIG. 10, an exemplary embodiment of the separation syringe 10 is provided, having many features in common with previously described embodiments, where like numbers refer to like parts, and the embodiment of FIG. 10 can be distinguished from the separation syringe depicted in FIG. 2 in at least the following aspects.

The particular embodiment depicted in FIG. 10 differs from the previously described separation syringe 10 in that it further provides at least one support element configured to prevent undesirable lateral movement or oscillation, that may manifest as wobble between one or more components relative to each other as the device is being spun to separate the fluid into fraction. Such oscillations or wobbles may negatively impact the creation of an interface between different fractions of the liquid being spun for separation within the separation syringe. As depicted in FIG. 10, the at least one support element may be a rod 50 positioned in the axial center of the syringe body 12, extended along the longitudinal axis. The rod 50 may be directed through the distal end of the syringe body 12, passing through the center of the cap 42 as shown. In such an instance, the pierceable septum 46 may be removed entirely, or may serve as a bushing that receives and conforms around the rod 50, or alternatively, the septum 42 may be repositioned to be off-center within the cap 42, but still generally axially aligned with the interior of the RBC/PRP separator wall 24, such that a needle, if so desired, may access the chamber 26 by being directed through the repositioned pierceable septum 46. The distal end of the rod 50 may further support the rotating separation syringe, where there may be provided a motor mount or a bushing that can mechanically engage with a device that facilitates the separation syringe 10 to be spun, as discussed herein. The rod 50 extends proximally, to be received in the center of the distal end of the PPP plunger 34, where the one-way valve 36 may conform around the rod, and yet still be capable of allowing fluid flow into the PPP containment chamber, as discussed herein. Alternatively, the valve 36 may be positioned alongside the rod, but still allow fluid flow into the PPP containment chamber 88. The rod may be any suitable material, such as metal, plastic, carbon fiber, and may optionally be treated to be bio-inert, or may be coated with a substance, such as may be useful to minimize interaction with the fluid being separated, or minimize the potential for clotting within the syringe, for example with an anti-coagulant.

In an embodiment, and as depicted in FIG. 10, the seal 52 may be an o-ring as shown and the annular ridge portion 58 may feature a tapered distal end around the outside rim of the distal end of the RBC/PRP separator wall 24, where such an inclined surface of the annular ridge portion 58 would be pressed against the seal 52. In this embodiment, as the RBC/PRP separator wall 24 is caused to move proximally, the tapered end of the RBC wall would slide relative to the seal 52, and smoothly open an annular opening 62 for the fluid passageway, as the tapered face gradually loses contact with the seal 52 when the RBC wall is urged proximally, leaving the seal 62 urged against the interior surfaces of the cap 42, and the outer wall 20. Subsequently, after the first fraction has passed through the annular opening, and into the outer chamber 22, as the RBC/PRP separator wall 24 is urged distally in order to close the annular opening 62, the tapered end of the RBC/PRP separator wall 24 would smoothly encounter and squeeze against the seal 52, and thereby gently close the fluid pathway, with increasing pressures against the seal 52. In this manner, the tapered surface would ensure that the annular opening is gently closed off to prevent further flow of fluid out from the inner chamber 26. Such a tapered end at the annular ridge portion 58 may be provided with an annular tapered surface that is presented with an angle that is less than 60 degrees and greater than 20 degrees; less than 50 degrees and greater than 25 degrees; greater than 30 degrees; less than 45 degrees; when measured relative to the longitudinal axis. In an embodiment, the tapered surface may have an angle that is from 30-45 degrees, when measured relative to the longitudinal axis.

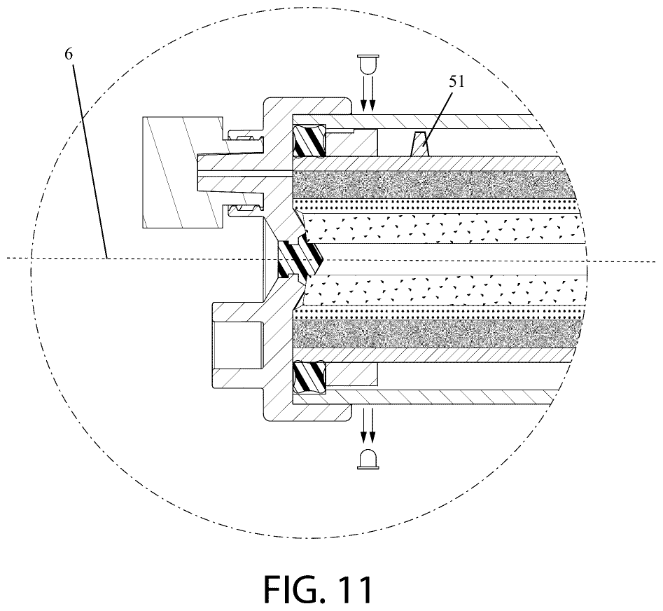

With reference to FIG. 11, the separation syringe 10 of FIG. 2 is shown, only with the additional provision of at least one support element, provided as support tabs 51. The support tabs 51 may be provided extending from the exterior surface of the RBC/PRP wall, extending outwards towards the inside surface of the outer wall 20, but still remain in a sliding relationship with the outer wall 20. The support tabs may be spaced around the perimeter of the RBC/PRP wall 24, ideally with equal spacing between the tabs 51 provided, in order to minimize the potential for wobble between components of the separation syringe as it is rotated. Such oscillations or wobbles may negatively impact the creation of an interface between different fractions of the liquid being spun for separation. In an embodiment, the support tabs 51 leave little clearance between the protruding end of the support tab 51 and the inside surface of the outer wall 20 (e.g., having less than 0.030 inches or having less than 0.020 inches, or between 0.010 and 0.005 inches) when the syringe 10 is assembled with the RBC/PRP wall 24 slidingly positioned within the outer wall 20. By controlling such clearance, the effect of any wobble that would occur is minimized by the tabs preventing the wobble from occurring to the extent that it would interfere with the separation process described herein, yet still allow sliding movement of the RBD/PRP wall. It is contemplated that placement of the support tabs 51 may be adjusted along the length of the RBC/PRP wall 24, through preferably positioned towards the distal half of the RBC/PRP wall. The support tabs 51 may alternatively be positioned about the protruding annular ridge portion, where such tabs 51 would be provided as narrow ridges fitting within the narrowed portion of the annular passage 62. In such an embodiment, the support tabs would also be spaced around the perimeter, with enough spacing between adjacent tabs 51, so as not to restrict or interfere with the flow of fluid through the annular opening 62, and further would not cause damage to cells or cause clogging as a biologic fluid fraction is caused to pass through the narrowed portion 68.

Aspects and use of the separation syringe according to various embodiments of the invention will now be described with reference to the figures. FIGS. 2-7 provide an illustration of the arrangement of the components of the separation syringe and illustrate the principle of operation of the embodiments of the devices according to the teachings below. Such teachings may be similarly understood with reference to the separation syringe 10 depicted in FIG. 10, where like parts are identified with like numbers, and serve similar functions, unless specified otherwise herein. As shown, in an exemplary embodiment, there is provided a multichambered syringe system 10 designed to separate biological connective tissues that are drawn into an interior chamber 26 of the multichambered syringe 10, for example, as depicted in FIGS. 1A and 1B. As depicted, the separation syringe 10 may be connected via any suitable connection on the distal cap 42, such as a luer fitting 44, to be in fluid communication with a needle inserted into a donor for obtaining a sample. The sample is drawn into the syringe body 12 as the plunger assembly 14 is retracted out from the syringe body, as is known with traditional syringe devices. The embodiment taught herein provides the ability for the biologic fluid mixture within the separation syringe 10 to be separated by specific gravity into three distinct fractions, that can then be physically isolated from each of the other fractions, all within the same syringe 10.

As shown, the multichambered syringe 10 is to be partially filled with biological connective tissues that are generally liquid, such as blood, adipose tissue aspirate, or bone marrow aspirate. The biological connective tissues provide a combination of constituent materials with differing specific gravities. With the biological connective tissue within the chamber, the device achieves separation of the constituent materials by having the multichambered syringe 10 being rotated about its longitudinal axis 6. The rotation is continued at a rotational speed, and duration that will cause the biological connective tissue to separate into distinct layers, or fractions, based on their specific gravity. It is anticipated that the rotational duration may be in the range of 30 seconds to 5 minutes and may be approximately 2 minutes in order to stratify the constituent materials into annular layers. The unique multichambered syringe provides benefit of allowing for radial gravitational separation of the fractions and, when needed, physical compartmentalization of the distinct constituent parts, fragments, or fractions. The embodiments herein allow the drawing of biological connective tissue directly into the multichambered syringe, as depicted in FIGS. 1A and 1B, or alternatively, the biological connective tissue may be drawn and delivered into the syringe from a blood sample that was collected and stored previously.

The multichambered syringe embodiments taught herein provide several advantages and improvements over previously known separation devices and methods, as will be discussed below. First, according to the embodiments taught herein, the biological connective tissue separation devices as taught herein allow for the biological connective tissue to be drawn directly into the separation device. The blood draw luer lock fitting 44 can be used to attach to a commonly used blood draw needle, butterfly needle with tubing, Jamshidi needle, Mercedes needle or similar medical devices. The plunger assembly 14 can be drawn back to accumulate the biological connective tissues within the inner chamber 26 of the separation syringe 10. To date, no such device (excluding vacutainer blood draw tubes) exists where a direct draw of biological connective tissue can be done directly into a device that will centrifuge, separate and be capable of re-administering final prepared biological connective tissues. Devices that are previously known in separation of biological connective tissue require a method of acquiring the biological connective tissue into such tools as syringes, prior to being transferred into device that would be used for separation. Each transfer step of the prior art separation devices introduces potential for contamination or inducing a coagulation cascade. Thus, previously known processes with prior art separation devices disadvantageously introduce a minimum of two transfers that potentially introduce shear forces that rupture cells during transfer through narrow orifices, as the biological connective tissue is being collected and prepared for use. As mentioned above, each transfer with prior art separation devices has the drawback of introducing a potential for contamination that could lead to such issues as infection.

Therefore, the embodiments described herein seek to improve upon the previously known separation devices that require a draw from a donor into a first container, which is then transferred into the prior art separation devices as known, which after the separation procedure of the sample, the desired fraction(s) is transferred to still another container for delivery or application to a treatment site. In contrast, the embodiments described herein allow drawing directly from the donor into the separation device 10, after the separation procedure, the same device may then be used to deliver the desired fraction to a treatment site, as will be explained.

Second, the same device 10 into which the sample is initially drawn, can then be rotated along its longitudinal axis 6, and the centripetal forces generated during rotation allow for separation of the biological connective tissue into specific gravity as distinct layers. The outermost layer (usually highest specific gravity) would be positioned against the RBC/PRP separator wall 24 with the inner layer (usually lowest specific gravity) would be positioned in the center or closest to central longitudinal axis 6 of the device, which each layer in the form of an annular ring. Varying volumes of the distinct constituent parts, fragments or fractions have been appreciated and understood to create a time dependency for separation. This separation can be manually or automatically controlled. Most products on the market function via a manual method where time is assumed based on a safety factor that would allow for worst case scenario for separation of the biological connective tissues. However, due to methods of separation, many of these products create shear in the sample, either thru the method of centrifugation (e.g., high speed acceleration, high pressure passage through narrow orifices) or via cell restrictive methods of separation (e.g., floating shelf systems limiting space between the outer wall and the floating shelf. Unlike previously known separation systems, the embodiments of the separation system described herein function via a soft start to avoid or significantly reduce shear forces that would be damaging to the biological connective tissue distinct constituent parts, fragments or fractions. Additionally, even with rotational rates that could generate high pressure, this flow of material through the annular passage may be modulated by the rate at which pressure could equalize through the vent 84, thus limiting the pressure differential between the inner chamber 26 and the outer chamber 22 as the annular passage is opened. Additionally, with the embodiments described herein, there are no added components or parts (such as with floating shelf systems) within the separation chamber that could induce unwanted shear. Furthermore, the embodiments taught herein rely on the gradual movement of the separated fractions of the samples during processing passing through annular passages that providing greater overall volumetric area within the passage, when compared to prior art devices using narrow discrete orifices that result in high speed ejection of the cellular materials. In the embodiment shown in FIGS. 4-4D, there are multiple stages where the flow rate might be affected, rather than just rely on a single restriction point. For example, a first stage of flow restriction may be found in the extent to which the annular opening 62 is created, by modulating the extent to which the RBC Actuator assembly 30 is moved proximally. Additionally, a second stage for flow restriction occurs as the cells pass over the ridge portion 58 and through the detector system beam 82. Neither of these stages would need to be of a size that would negatively impact the cellular viability as the fraction passes through the annular passage, and in cooperation each would affect the flow rate, along with controlling the rate of pressure equalization to allow transferring the first fraction from the inner chamber 26 to the outer chamber 22 in a manner that preserves cellular viability. In contrast, previously known separation devices have been described utilizing one or more small discrete openings that serve as rate limiting outlets that could easily become blocked during processing. Additionally, these prior art devices relied on ejection of a fraction into an open space, as the fraction is ejected under great pressure from an orifice during high speed centrifugation, which will lead to impact of the cells within that fraction against a containing wall opposite the orifice, with such impact destroying many of the cells in the ejected fraction, and negatively affecting the viability of the cells within that fraction. Unlike previously known separation systems, the embodiments of the separation system described herein rely on decanting the first fraction past a protruding ridge portion 58, with the rate of fluid flow being limited by a variety of manners using multiple control methods (including controlling the extent of opening of the annular passage 62, the characteristics of the ridge portion 58 affecting the flow rate, and the ability to modulate the air pressure equalization through the vent 84 between outer chamber 22 and inner chamber 26). In this manner, unlike prior art where cells were subjected to an impact against a containing wall as they were ejected from the prior art separation chamber, utilizing the embodiments described herein, the cells when processed are decanted from the inner chamber into the outer chamber, without being subjected to harsh physical impacts to the cells when travelling from the inner chamber 26 into the outer chamber 22, nor are the processed cells required to pass at high speed through narrow orifice openings that may negatively impact the cells' viability. In this manner, the embodiments described herein are able to preserve the cellular viability of the processed cells, for example, those cells that are isolated within the outer chamber. Additionally, the viability of the cells remaining within the inner chamber and the PPP plunger chamber will also be preserved, as will be discussed.

It is anticipated, that with these improved features and the reduction of shear forces, that this system may avoid the need for, or significantly reduce the need for introducing an anticoagulant, such as ACDA or Heparin.

Third, decantation of designated constituent parts, fragments or fractions, via an opening provided as an annular passage 62 extending entirely about the perimeter, with the decantation forces created by movement of the RBC/PRP separator wall 24. Providing an annular passage 62 for decantation of the first fraction significantly increases the functional area for evacuation, thereby minimizing risk of blockage during processing. This annular passage is an improvement over systems that utilize provide discrete openings, whether as a single discrete outlet, or a plurality of discrete opening outlets, as such systems suffer from blockages during processing that can happen due to clotting of the biological tissue, that during drainage can obstruct the discrete hole/port outlets of other previously known devices. Additionally, these types of systems suffer from vortices, during decantation, that lead to mixing of the separated components during the removal process, as the interface between layers can be disrupted by the exiting flow. With prior art devices, it has been observed that where the sample is blood, especially from a patient who is suffering from dehydration, there is a propensity for increased viscosity in the blood sample take, this leads to blood clotting and difficulty in previously known systems for centrifugal separation of the tissue's distinct constituent parts, fragments or fractions. In contrast, the annular passage opening of the embodiments of the devices described herein do not suffer from these concerns, due to providing greater surface area and increased length of the passage, such that the controlled opening of the annular passage aids in removal of the higher specific gravity distinct constituent parts, fragments or fractions without damage to cells or being subject to clogging of the annular passage 62. The controlled removal can be automated via a LED control system 82 for monitoring the processing during the RBC separation. In an embodiment, there is provided a light source and photosensor near the distal end of the RBC/PRP separator wall 24, with the light beam passing through at least a portion of the annular passage 62. This allows for automating the decanting step and controlling the removal of the first fraction to a precise pre-determined value, as measured by the optical sensor. Such a sensor, upon detecting the desired value, could provide feedback for triggering the closure of the RBC drain assembly 28 acting as a valve to block the fluid movement through the annular passage 62. For example, where the device is separating a sample of whole blood into its constituent fractions, then decanting with the above-described automated system, such a system would allow adjustment to varying degrees of hematocrit (red blood cells). Generally, a normal range is considered to be, for men, 38.3 to 48.6 percent hematocrit and for women, 35.5 to 44.9 percent hematocrit. For children ages 17 and younger, the normal range varies by age and sex. These ranges create an inherent problem with existing systems that either needs to acquire an average value or require a relative manual method of decantation (decreasing accuracy and consistency of final product). The system described herein has over-come the need to manually control the operation of the valve closure, where such operation is reliant upon visual control, for example where the user optically monitors the interface of the RBC portion. Manual control is reliant upon user skill and attention, reflexes, and user discretion in determining the interface location, for example, positioning the interface between the red blood cells and the Platelet Rich Plasma at the appropriate location to maximize the collection of the desired fraction. Such user involvement introduces user subjectivity, or error, or lapses in timing for valve operation. In contrast, an automated system as described herein, utilizing an optical detection system 82 to trigger valve closure of the RBC drain actuator 28 would increase the precision of decanting control. The process has a feedback system of control via RBC air vent 84 and seal (venting system) that allows for self-regulated flow at the annular opening 62 between the RBC/PRP separator wall 24 and the distal cap 42, controlled by a feedback system of the LED sensor 82 controller and driven by mechanisms such as a throw out bearing or solenoid driven yoke and bearing 110, that can open and close the RBC separator assembly 28 (during rotation of the device). The RBC separator assembly 28 may be mechanically driven, acting upon a triggering signal provided by the LED sensor system 82 and a controller, for example, a microprocessor may be utilized to monitor the data sensed by the LED sensor system 82, and at a desired threshold value may initiate a mechanical movement 110 acting upon the RBC slider drain actuator 28, thus directing the opening and closing of the RBC actuator 28 via LED controller 82 feedback, to selectively block or allow decanting of the first fraction of the sampled connective tissue into the outer (RBC) chamber 22. This process can be conducted at varying timing of the centrifugation step, examples such as at the initiation of the centrifugation, at the end of timed centrifugation or anytime during the process to control the material that is decanted into the RBC outer chamber 22. The control of the material decanted can be calculated, for example, based on the material, volume, centrifugal forces generated and timing, allowing for preparation of specified biological connective tissue distinct constituent parts, fragments or fractions.

The rate of decantation of the first fraction into the RBC outer chamber 22 can be controlled by a variety of factors, including: 1) the rate at which the separation device is spinning, where higher rotational speed will create stronger centripetal force urging the first fraction outwards from the central axis; 2) the extent to which the RBC slider drain actuator 28 is actuated will determine the amount of clearance between the distal seal 52 and the distal end of the RBC/PRP wall 24 (as depicted in FIG. 4A), with greater actuation of the RBC slider drain 28 creating greater clearance through the annular passage 62 and allowing less restricted flow of the first fraction and a faster rate of decantation; 3) the shape and dimension of the protruding annular ridge portion 58 can control the flow characteristics of the first fraction, as well as define the dimensions of the narrowed annular passage 62, such as the thickness and length of the narrowed annular gap remaining between the outermost surface of the annular ridge portion 58, and the inside surface of the outer chamber wall 20; and 4) controlling the dimensions and characteristics of the vent opening 84, where small openings would slow the rate of pressure equalization between the outer chamber 22 and the inner chamber 26, which indirectly limits the rate of decantation of the first fraction into the outer chamber 22. Any one of these factors may be adjusted alone, or in combination, to provide a desired maximum rate of decantation of the first fraction.

Fourth, the embodiments of the separation device 10 taught herein provide for physically separating the fractions remaining in the inner chamber 26 after decanting the first fraction into the outer chamber 22. As the rotation of the syringe device 10 is maintained during the separation, the remaining layers within the inner chamber 26 will remain as separated annular layers, as depicted in FIG. 5A, with the PRP layer residing against the interior of the RBC/PRP separator wall 24, and the PPP layer arranged closest to the central longitudinal axis 6, next to the air core in the center of the interior chamber. As shown, the PRP and PPP interface, between the PRP layer and the PPP layer, is depicted immediately to the outside of longitudinal alignment with the border between the PRP plunger 32 and the PPP plunger 34. The separation syringe embodiments then provide for separation and isolation of each of the remaining fractions, for example, through the use of concentric chambers that are created by advancing the components of the plunger assembly 14. The plunger assembly 14 is capable of functioning as a single unit (such as when drawing the sample into the inner chamber 26 or as two separate units when isolating the remaining fractions, using the PRP plunger 32 and PPP separator plunger 34 independently, depending on the step within the separation procedure. A centrally located plunger is provided as the PPP separator plunger 34, which is positioned over the longitudinal axis 6, and is capable of being advanced or retracted into the syringe body 12 by moving in a direction parallel to the longitudinal axis 6. The PPP plunger 34 may be driven during centrifugation by a mechanism, such as a lead screw, that will drive the PPP separator plunger 34 without advancing the PRP plunger 32. During the travel of the PPP separator plunger 34, the central most biological connective tissue will pass through the flap valve 36 and be captured in the center chamber 88 of the PPP separator plunger 34 while leaving or physically compartmentalizing the remaining biological connective tissue between the RBC/PRP separator wall 24 and the PPP separator plunger 34. Being able to conduct these steps during centrifugation should not be trivialized, especially since known prior art systems try to tip and pour this biological connective tissue, shear past a floating shelf or use discrete holes/ports to eject fluid fractions, with the drawback of creating vortices during the removal process that interrupt the laminar interface between fractions. The various embodiments described herein provide an advantage in processing in that the processing steps overcome these issues with the prior art devices, as the embodiments described herein maintain the sample in rotation during centrifugation, and while leveraging the centrifugal forces and internal pressures to maintain the separation of the layers of the biological connective tissue as distinct fractions. Furthermore, the embodiments taught herein are capable of capturing the central most fraction of the biological connective tissue, so that it is to be cleanly isolated within the PPP plunger chamber, with minimal or no disturbance of the remaining biological connective tissue.

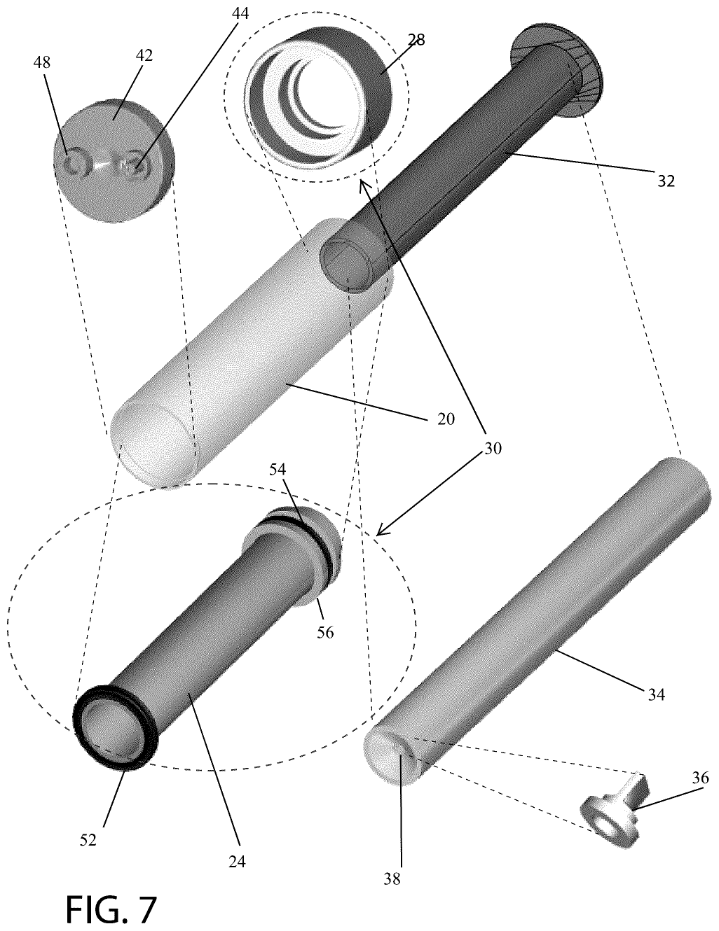

Finally, with centrifugation and initial steps complete, the device 10 can be removed and used to readminister final biological connective tissue via the advancement of the PRP plunger 32 to force the remaining fraction contained within the interior chamber 26 out from the luer fitting 44 and allowing for the delivery of the final constituent parts, fragments or fractions back to the patient, all within in a single device. The remaining biological connective tissue fraction contained in the inner chamber 26, now limited to the area between the between the RBC/PRP separator wall 24 and the PPP separator plunger 34, and in this embodiment, is the desired portion of the biological connective tissue that can be used in many clinical applications. Additionally, the biological connective tissue that was captured in the PPP chamber 88 within the PPP separator plunger 34 can be extracted via the distal most end of the syringe body 12, via the pierceable septum 46 provided in the distal cap 42 and may be used as medically necessary. In still another embodiment of the multichambered syringe, and with reference to FIGS. 8A and 8B, the separation syringe 10, as previously described can be used to separate the biological connective tissue into fractions by specific gravity, similar to the embodiment of FIGS. 2-7. This embodiment utilizes, for the most part, the same components only, rather than rely on actuation of the previously described RBC drain actuator 28, the device 10 instead provides a distal seal 52 that serves as an overcomeable valve. As before, the distal seal 52 is provided at the interface of the RBC/PRP wall 24 and the endcap 42. In this embodiment, the seal 52, as above, is placed at the distal end of the outer chamber wall 20 against the distal cap 42. The distal seal 52 also seals against the distal end of the RBC/PRP wall 24, as shown in FIG. 8B, which is permanently spaced apart from the distal cap by approximately the dimension of the distal seal 52, as shown. This embodiment obviates the need for the RBC/PRP separator wall 24 and RBC actuator assembly 28 to move by being translated axially in order to create an opening for fluid flow as has been previously described. Instead, the embodiment provides the distal seal 52 as an overcomeable valve that would remain sealed, as depicted in FIG. 8B, until a sufficient threshold pressure is created by the centrifugal force of rotation of the separation syringe 10 around the longitudinal axis 6, in the manner described previously. Once the threshold pressure is achieved, the overcomeable valve's cracking pressure is met, and at least a portion of the distal seal 52 will deflect to allow fluid flow through an annular passage 62, as shown in FIG. 8A. In such an embodiment, with the cracking pressure for the distal seal 52 achieved by rotational force urging the contents of the inner chamber against the distal scal, and the exit pressure of the first fraction is able to deform a portion of the distal seal 52 to create a slight opening, and which would be an annular opening extending around the circumference of the RBC/PRP wall 24 distal end, as depicted in FIG. 8A, between a fixed RBC/PRP wall 24 and the endcap 42, to allow fluid flow past the deformed seal 52. For example, in an embodiment, equalization of pressures within the chambers may be accomplished using a vent 84, as previously discussed, but would utilize a one-way valve on the vent to limit the direction of air flow for the pressure equalization through the vent 84 in a direction of travel from the outer chamber 22 towards the inner chamber 26. Such a valve would prevent the movement of fluid through the vent opening 84 in the reverse direction as the separation syringe 10 is being spun to establish the stratification of fractional layers of the sample. Alternatively, another overcomeable valve may be provided to allow venting and equalization of pressures between chambers. Such an overcomeable valve may allow venting pressures to equalize when a sufficient cracking pressure is met to deform the seal, in a manner similar to that described previously. To ensure that the annular separation of the fractions is established before achieving the cracking pressure and initiating the fluid flow, the separation system 10 may be spun at an initial rate to establish the separation of the fractions as annular layers. Once the separation of fraction layers is complete within the inner chamber 26, the rotational speed of the separation syringe can then be increased to generate enough pressure and overcome the seal's cracking pressure, whereupon the first fraction of the biological connective tissues can flow past the overcomeable valve and through the annular passage 62 as depicted in FIG. 8A and pass through the annular narrow passageway into the outer chamber. As before, a light detection system 82 may monitor one or more characteristics of the first fraction as it passes through the light beam at the narrow annular opening 62. Once the interface of the first fraction (having the highest specific gravity) and the next fraction (having the next highest specific gravity) is detected, the centrifugation system will be caused to reduce rotational speed of the syringe 10, whereupon the cracking pressure of the seal 52 is no longer met, and the distal seal will again close off the fluid flow out from the inner chamber 26. The rotation of the syringe 10 is to be continued to maintain the separation of the remaining fractions within the inner chamber 26, such that the plunger assembly 14 can be advanced, as described previously, to isolate the second fraction (having the lowest specific gravity within the PPP plunger 34. Once the third fraction is physically isolated from the first and second fractions, then the centrifugation may be halted, and the separation syringe 10 prepared for delivering the third fraction to a treatment site, as has been previously described. As above, if there is a need to utilize the second fraction (the lowest specific gravity fraction) from the PPP plunger 34, access can be obtained by directing a needle of a separate syringe through the septum 46 provided in the center of the distal cap 42 of the separation syringe 10, passing further through the duckbill valve 36, in order to access the contents within PPP chamber 88 of the PPP plunger 34.

The method of using an embodiment of the invention to process a sample within the separation syringe will be described with reference to FIGS. 1-6 and 10.

With reference to FIGS. 1A and 1B, a sample of biological fluid is drawn into the inner chamber 26 of the separation syringe 10. This may be accomplished by any suitable technique as will be known to those of skill in the art. For example, as shown in FIG. 1A, a butterfly needle and tubing may be utilized for blood collection from a living being serving as a donor. The collection method may follow standard blood collection protocols for insertion of a butterfly needle into a vein. Alternatively, a sample may be taken from a stored biologic fluid, such as from a blood bank. It is envisioned that the separation syringe may be utilized with anti-coagulated or non-anticoagulated biological fluid. The sample may be drawn into the center inner chamber 26 of the concentric barrels through a luer lock fitting 44 or similar type of opening connected to a standard blood collection needle. This is done by drawing back the plunger assembly 14 (PRP plunger 32 and PPP plunger 34) which creates a negative pressure within the center chamber 26 of the concentric barrels. When the separation syringe 10 is appropriately filled with the desired volume of biological fluid, the separation syringe may then be disconnected from the needle, Jamshidi needle, etc. and the luer fitting capped. The separation syringe may be used to process a variety of sample volumes, for example, approximately 30 mL, or 60 mL. It is recognized that using the teachings herein, the separation syringe may be scaled up or down to accommodate varying sample sizes for processing.

If so desired, the inner chamber 26 may optionally be treated with, or contain an anti-coagulant solution or mixture within the inner chamber. In an embodiment, the anti-coagulant may be previously introduced as an aerosol mist, or ultrasonic mist, or a vapor of a solution containing concentrated anti-coagulant, that can be introduced into the inner chamber, and allowed to dry on the inner walls of the inner chamber. This dried anti-coagulant may store for an extended period of time before use without experiencing significant degradation. As the sample volume is introduced into the inner chamber, the sample volume will solubilize the anti-coagulant from the inner chamber walls and provide anti-coagulated sample for processing. As is known, an anti-coagulated sample may beneficially avoid the occurrence of premature clotting of the sample, during processing or use.

Alternatively, where the sample is drawn directly from a patient, the manner of processing, and the duration of time the sample is to be processed is of a suitable short duration, such that it is possible to collect, process and apply the isolated fraction to a treatment site in a manner that does not require the use of an anti-coagulant. It is anticipated that the device will operate similarly, wither coagulated sample or anti-coagulated sample.

Once the sample is drawn into the inner chamber 26 of the separation syringe 10, as depicted in FIG. 1B, the butterfly tubing needle may then be disconnected, and a cap applied on the luer fitting 44 to prevent leaking of the sample and preserve sterility.

The separation syringe unit 10 containing the sample volume, as depicted in FIG. 2, is then ready for separation processing. As shown, the discrete sample volume is entirely within the inner chamber 26 of the separation syringe 10, the plungers of the plunger assembly 14 are retracted, and the RBC Drain actuator assembly 28 is provided in the closed position. The syringe 10 may then be placed secured for use in the centrifuge unit (not shown), by first being properly aligned for placement in the centrifuge unit which will lock the syringe into the system to allow spinning of the syringe, and additionally monitor the data from the light detection system 82 and initiate the actuation of the RBC drain actuator 28 and/or the linear drive for the PPP plunger 34 at the appropriate time. The motor for the centrifuge is actuated, for example by pressing a “start” button to initiate the rotation of the centrifuge and initiate the automated processing of the sample. The separation syringe 10 then is rotated axially to create a centrifugal force acting on the biological fluid mixture within the inner chamber 26, and thereby separating the sample into its components by specific gravity. The initial spin is at a rate of rotation that is adequate to separate the fractions by specific gravity within a short period of time, for example less than 120 seconds. The rotation of the syringe 10 unit is reflected in FIG. 3A, by the circular rotation arrows at the distal end of the syringe body 12. Additionally, FIG. 3A depicts the directional movement for the RBC drain actuator 28, reflected in the vertically opposing arrows to indicate axial translation of the RBC Drain Actuator assembly 30, as will be discussed. With reference to FIG. 3B, in the instance of separating blood (a biological fluid), the red blood cells would separate closest to the inside wall surface of the inner chamber 26 as the fraction with the highest specific gravity, forming the outermost layer. The white blood cells lining the red blood cell layer toward the center, followed by the platelets, and then plasma forming the innermost layer closest to the longitudinal axis, as the fraction with the lowest specific gravity. The sample may provide an air core at the central axis 6. The stratified annular layers can be seen in detail with reference to FIG. 3B, with the entire volume contained within the inner chamber 26 and separated into fractions as annular layers. Separation of layers would be performed similarly with the embodiment depicted in FIG. 10, with like parts having like numbers, and serving the same roles. In other words, the centrifugation yields concentric stratified constituent annular layers of the mixture, with each of the adjacent concentric stratified constituent layers defining a mixture interface or boundary between adjacent layers. After an initial centrifugation period of a duration of time adequate to create the separated fractional layers, e.g., approximately 120 seconds or less, the RBC drain actuator 28 may then be triggered, whereupon the RBC/PRP separator wall 24 and RBC actuator assembly 30 is moved from a closed position, depicted in FIG. 4, to a set position in which an annular opening 62 is created between the RBC/PRP separator wall 24 and the distal seal 52 and end cap 42, with activation of the LED control system 82, and as depicted in FIG. 4A. This concentric radial opening 62 communicates with the layer of red blood cells against the inside wall of the inner chamber 26. The first fraction (e.g., red blood cells) will exit the inner chamber 26 through this radical opening 62 due to pressure generated by the centrifugal force and flow into the outer chamber 22. As red blood cells exit the inner chamber 26 and are decanted past the narrow passage over the ridge element 58, the volume of fluid entering the outer chamber from the inner chamber is replaced by air exchanged from the outer chamber 22 (the RBC chamber) and venting into the inner chamber 26 via a vent 84 exposed by the movement of the RBC/PRP separator wall 24 and RBC actuator assembly 30. The vent 84 can be seen in the wall of the RBC/PRP wall 24, adjacent to the proximal seal 54, while the RBC actuator 28 is in the closed position. The vented air forms a column in the center of the inner chamber 26 that grows larger as more volume of RBC is decanted past the ridge portion 58 and air is vented into the inner chamber 26. It is also conceived that without a vent, that continued rotation and evacuation of the red blood cells will result in a vacuum core being formed, as the blood is degassed and possibly drawing vapor from the liquid due to the reduced pressure at the center of rotation. After a calculated amount is received within the outer chamber 22; or alternatively after the majority of the RBC fraction is decanted into the outer chamber, as monitored by the LED control system 82, the light detection system will recognize that all or substantially all of the red blood cells have been decanted into the outer chamber, as depicted in FIG. 4C, at which point, the RBC/PRP separator wall 24 and RBC actuator assembly 30 is triggered to be moved back to the original position, as shown in FIG. 4C, thus closing the concentric radial opening as depicted in FIG. 4D. The concentric radial opening 62 should be closed before the layer of platelets in the volume within the inner chamber 26 can pass through the annular radial opening 62. In FIG. 4D, the red blood cells are preserved in the outer chamber 22, and the remaining volume including the PRP and PPP layers are retained within the inner chamber 26.

Once the RBC drain actuator 28 is returned to the original, closed position, while the rotation of the syringe 10 continues, as depicted in FIG. 5A, the PPP plunger 34 is ready to be advanced. As shown, the inverse conical leading end of the PPP plunger 34 provides an annular leading edge that will be aligned with the inside edge of the innermost fraction. As depicted, the outer diameter of the PPP plunger 34 is precisely designed to funnel the plasma along the leading edges tapered conical design, through the entrance port 38 and flap valve 36 and into the PPP plunger chamber 88. Thus, the PPP plunger 34, as it advances, will have the outside surface of the PPP plunger 34 generally aligned with the interface of the middle fraction (the PRP) and the innermost fraction (the PPP). The advancement of the PPP plunger 34 into the inner chamber 26 is depicted in FIG. 5B, with a portion of the PPP fraction being received within the hollow interior chamber 88 of the PPP plunger 34, by passing through the flap valve 36. As the plunger assembly 14 is rotating with the contents of the inner chamber 26, the advancing PPP plunger 34 does not disrupt the annular layer of the PRP fraction, and the PPP fraction is forced towards the entrance port 38 at the peak of the conical leading edge as the PPP plunger 34 is fully advanced toward the distal cap 42. The advancement of the PPP plunger 34 may be performed as part of a timed sequence, initiated upon the closing of the annular passage 62 as the RBC/PRP separator wall 24 is urged to the initial closed state against the distal cap 42. For example, the centrifuge system may actuate a screw drive system, that advances the PPP plunger 34 into the interior chamber 26, through the proximal end cap of the RBC Drain Actuator 28. The driving motion creates a pressure in the inner chamber 26 and forces the PPP fluid through the flap valve 36 into the PPP chamber 88 within the PPP plunger 34 until fully depressed (in contact with the end cap 42), as depicted in FIG. 5C, where substantially all of the innermost fraction (the PPP) has been urged through the flap valve 36 into the PPP chamber 88, and leaving the middle fraction, such as the PRP, left in the space remaining between the exterior of the PPP plunger 34, and the interior surface of the RBC/PRP wall 24. After capturing substantially all of the PPP within the chamber 88, the mechanism for advancing the PPP plunger may be reversed, leaving the PPP plunger in the advanced position.

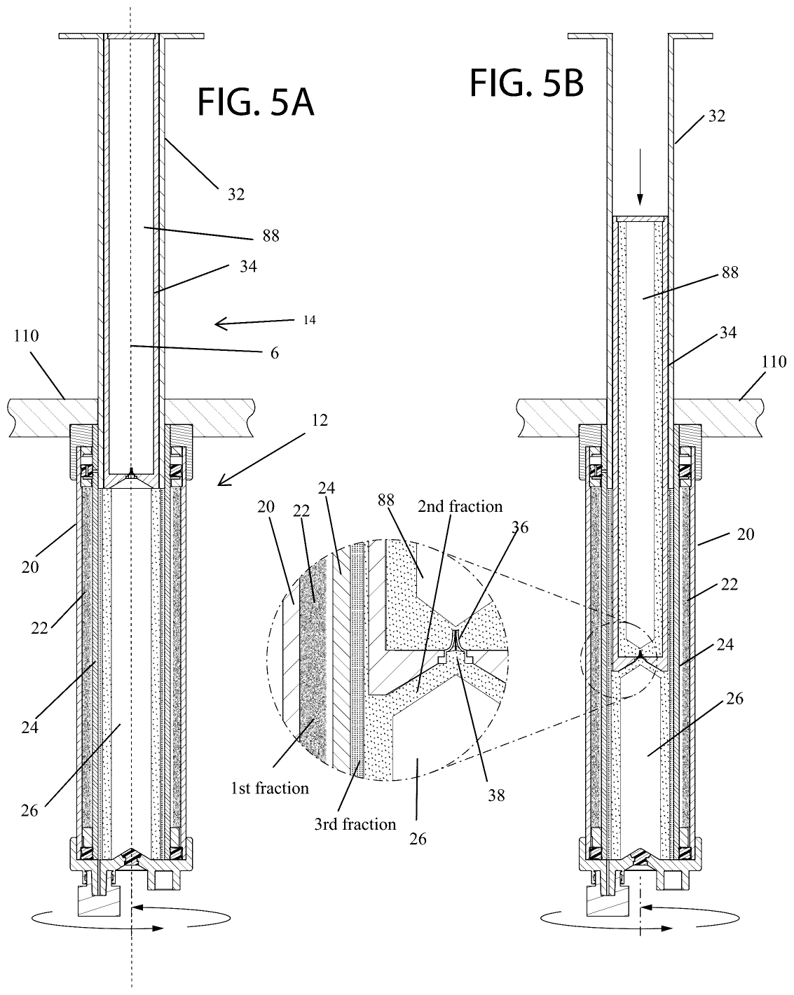

The rotation of the separation syringe 10 may then be halted, the centrifuge powered down, and the syringe may be removed from the centrifuge system. At this point, the platelet concentrate within the inner chamber 26 remaining in the space between the PPP plunger 34 and the PRP wall 24, is ready for use, as depicted in FIG. 6. In order to deliver the platelet concentrate to a treatment site, the cap on Luer fitting 44 can be removed, and optionally, a needle may be placed onto the ejection luer fitting for the ejection of the platelet concentrate. The luer fitting 44 for ejection of the PRP is aligned with the region where the PRP will be isolated within the inner chamber 26. Advancement of the PRP plunger 32 will force the concentrated biological fluid out the ejection luer fitting 44. The PRP plunger 32 is dimensioned to fit within the space remaining between the outside edge of the PPP plunger 34 and the inside edge of the RBC/PRP wall 24, and substantially ejects all of the platelet concentrate when the PRP plunger 32 is fully advanced in a distal direction against the distal end cap 42.

Optionally, there may be a need to collect or administer the fraction contained within the chamber 88 inside the hollow PPP plunger 34. This could be achieved by inserting the needle of another syringe through the central septum 46 of the distal cap 42 and advancing the needle through the flap valve 36 to enter the PPP plunger chamber 88. The syringe may then extract the PPP fluid as will be familiar to those of skill in the art. The syringe needle may be removed from the separation syringe, and the contents of the syringe are ready for storage or administration, as appropriate.

Optionally, there may be a need to collect or administer the first fraction contained within the outer chamber, after the separation and isolation process described herein. Once the third fraction (e.g., the PRP) has been removed from the inner chamber 26, access to the first fraction could be achieved by first withdraw the plunger assembly a small amount in order to create fluid communication between the septum 46 and the distal seal 52. Subsequently, the RBC drain actuator assembly 30 should be retracted at least partially by being moved in a proximal direction, in order to open the annular passage 62. A needle on a standard syringe may then be inserted through the septum 46 and advanced a small amount into the inner chamber 26. A vacuum drawn by withdrawing the plunger of the standard syringe will draw the first fraction out from the outer chamber 22, passing through the annular passage 62 into the inner chamber (the reverse of the flow described previously), where the sample of the first fraction could be accessed the needle in the inner chamber to draw the sample into the standard syringe.

In another embodiment, the system uses an overcomeable valve as part of the distal seal 52 at the interface of the RBC/PRP separator wall 24 and the endcap 42. The overcomeable valve aids in the isolation of the red blood cell layer. Thus, once the initial spin has occurred causing the isolation of the blood components into separate layers, the rotational speed can increase, generating increased pressures within the inner chamber 26. The overcomeable valve of the distal seal 52 will be set to open at this preset increase pressure, allowing for the passage of the red blood cells into the RBC collection outer chamber 22. The LED control system 82 will function to identify when the optimal volume of red blood cells are removed, thus activating the slowing of the rotational speed and thus reduction of the internal pressure, whereupon the annular passage 62 will be sealed again by the distal seal 52.

In another embodiment, the syringe body 12 can be made of a clear (transparent) material so that the progress of the red blood cell removal can be observed via other non-LED based control systems. This can allow for precise timing for controlling the opening and closing of the annular opening 62 to end the exiting of the red blood cells.

Another embodiment accomplishes the concentration through precise timing of the opening/closing sequence and the starting and stopping of the motor.

In another embodiment, the system may feature a reusable drive component with a motor that is arranged to be coupled to a disposable separation syringe 10 component, wherein the blood products are centrifuged, separated, and contained entirely within the disposable syringe 10, such that the drive component is not exposed to blood product and may be reused without fear of contamination.

In another embodiment, the separation syringe unit may be optimized to process biological fluids such as bone marrow to isolate cellular components such as mononucleated cells.

In another embodiment, the separation syringe unit may be optimized to process biological fluids such as blood to isolate cellular components such as endothelial precursor cells.

In another embodiment, the separation syringe unit may be optimized to process biological fluids such as blood to isolate plasma proteins components such as alpha-2-macroglobulin.

In another embodiment, the separation syringe unit may be optimized to process biological fluids such as adipose tissue to isolate cellular components such as adipose derived stem cells.

In another embodiment, the separation syringe unit may be optimized to process biological fluids such as adipose tissue to isolate cellular components such as stromal vascular fraction (SVF) of adipose tissue known to contain mesenchymal stem cells (MSC), T-regulatory cells, endothelial precursor cells, preadipocytes, as well as anti-inflammatory M2 macrophages.

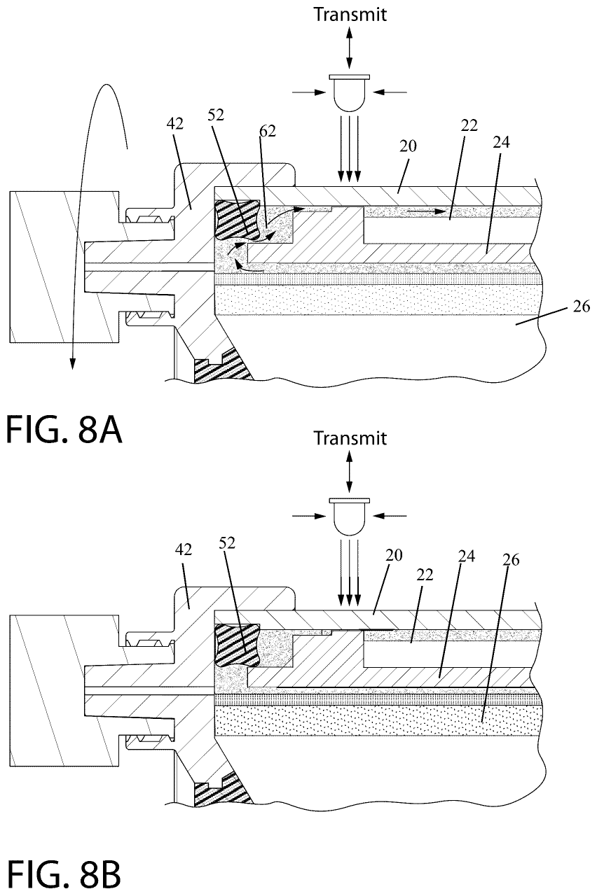

In another embodiment, as depicted in FIG. 9, a version of the separation syringe can be used to separate the biological connective tissue. This embodiment, utilizes the same parts above however incorporates an improvement to the syringe tip, or distal end cap 42′ designed to maintain the uniqueness of an annular design where a cone (cones) 120, as annular orifices, is spaced appropriately in the PRP/PPP funnel to accommodate the distinct layers created in the body of the syringe. This embodiment functions similarly that initial embodiment, however allowing either during or post centrifugation, the isolated biological connective tissues can flow out of the annular orifices into separate containers to maintain the isolated layers in storage containers until needed for use. Such a syringe may be utilized, for example, by drawing a sample from a donor into the syringe body and centrifuging the syringe 10′ as has been previously described. Once the stratification of the sample has occurred, a traditional plunger as known from the prior art, may be advanced distally, whereupon the stratified layers would be urged towards the funnel tip providing multiple annular openings (A, B, C) aligned to receive the desired fraction within each of the tip openings, as can be seen with reference to FIG. 9. Once the plunger is fully advanced into the syringe body 12′, the fractions will be separated within a chamber associated with only one of each of the annular opening in the tip or distal cap 42′. Once the syringe is removed from the centrifuge device, each of the chambers may be accessed as needed to retrieve the desired fraction for further use.

While the embodiments described above reference components by names associated with fluid components associated with an exemplary application where the biological fluid mixture is whole blood, such as those identifying components or associated with fractions, such as the components referred to as the PPP plunger, RBC Actuator/RBC/PRP Wall, and PRP plunger, it is recognized that the nomenclature used to identify component isolated by the device need not be limited only to applications of whole blood fluids, but rather by the fraction isolated or acted upon by the component. It is thus recognized that the PPP plunger could also be appropriately referred to as the plunger for the fraction with the lowest specific gravity (e.g., the first fraction); the PRP plunger could also be appropriately referred to as the plunger for the fraction with an intermediate specific fraction (e.g., the second fraction where the mixture is separated into three fractions), and the RBC Actuator/RBC/PRP Wall could appropriately referred to as the actuator for allowing the separation of the fraction with the highest specific gravity (e.g., the third fraction), with the identified wall separating the highest specific gravity fraction (the RBC) from the intermediate specific gravity fraction (the PRP). One skilled in the art will recognize that operation of the device will perform similarly for separation of fluid fractions from a biologic fluid mixture and is not limited solely to application for separation of whole blood into PRP, PPP, and RBC, but could be similarly employed for any fluid mixture capable of being separated into fractions by centrifugation about the longitudinal axis of the syringe assemblies described herein.

It is also recognized that, using the teachings herein, one skilled in the art would understand the necessary actions or modifications to the operation or to the device in order to isolate a fluid mixture into more or less than the three fractions discussed above with reference to whole blood. For example, providing additional concentrically arranged plungers, or providing further additional actuatable valves with channels that lead to concentrically arranged chambers (by providing additional concentrically arranged walls around the inner chamber) could allow for separation of more than three fractions from a fluid mixture. Alternatively, isolation of two fractions could be performed solely through the operations of dual plungers arranged within an inner chamber, as will be discussed with reference to the syringe assembly 10′ below.

In an exemplary application, the centrifuge separation device could be utilized to isolate a first fraction of a sample from a second fraction of a sample. As a non-limiting example, a biologic fluid containing a fraction of viable, live cells could be separated and isolated from a fraction containing dead cells or cellular debris using the syringe assembly 10′, such as by centrifuge separation of a biologic fluid mixture containing a mixed population of cells, such as a suspension of cells. It is known that the separation of dead cells from live cells could be performed by centrifugation, where density-gradient centrifugation is performed by spinning a heterogenous mixture at high speed, allowing fractions to separate by density, as has been previously described. The separation of viable cells from dead cells and cellular debris relies on the principle that dead cells and cellular debris, when the cell suspension is centrifuged, will tend to separate from the live cells due to the centripetal force applied by rotation about the longitudinal axis, to form a first concentric layer with the live cells, and a second concentric layer with dead cells and cellular debris.

In an embodiment, the centrifuge separation device would be provided to quickly and reliably isolate a fraction containing live cells from fraction containing dead cells from a biologic fluid mixture containing both live and dead cells. Such an embodiment could be usefully applied to enrich the population of live cells from a fraction of a biologic fluid mixture, such as a sample of semen as may be used for artificial insemination. Such an application could be beneficially employed in the field of artificial insemination for breeding animals, such as racehorses or livestock, or even may be useful for human artificial insemination practices, where enriching the percentage of live sperm cells in a provided sample (be separating out the dead, non-viable cells, to isolate a sample containing a larger proportion of live cells, would increase the likelihood of successful fertilization, and reduce the need for follow on attempts at artificially inseminating a breeding female. Such centrifugation should be rapid to prevent further cell degradation, yet still minimize physical harm or shear forces applied to the cells as may occur by passing through narrow constrictions that might otherwise rupture the living cells.

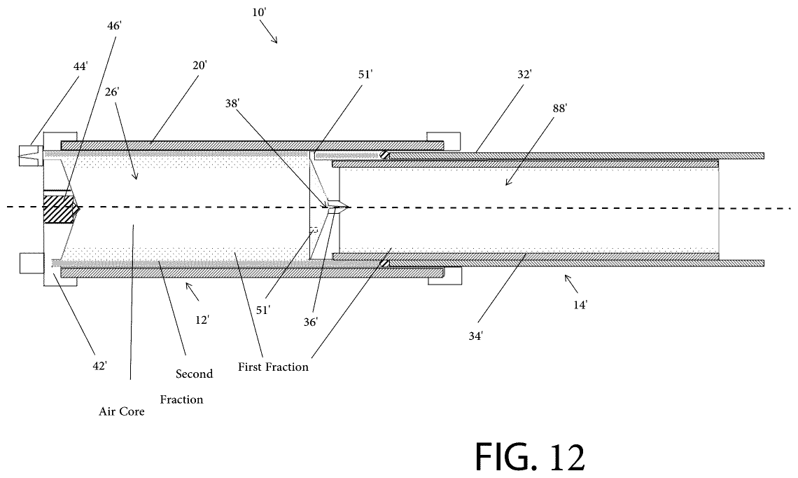

In such an embodiment, the heterogenous sample collected could be inserted into the inner chamber 26 of the provided separation syringe 10, as discussed previously. As there is only a need to isolate a first fraction from a second fraction, there would be no need to separate the sample into three isolated fractions as previously described, thus, there would be, for example, no need to provide, nor actuate the previously described RBC slider drain actuator 28, and there would be no fraction introduced into an outer chamber 22. Rather, the provided sample may be spun within the separation syringe 10′, as the syringe body 12 is caused to rotate rapidly about its longitudinal axis, as has previously described, and thus separate the fluid sample (e.g., a biologic fluid such as a semen sample) into concentrically stratified fractions, forming at least a fraction of higher specific gravity positioned closer to the outside boundary of the inside chamber 26′ of the syringe body 12′, to be positioned as an annular layer against the interior wall of the inner chamber 26′ (e.g., forming an outer concentric layer), as the syringe assembly 10′ is caused to rotate about its longitudinal axis. Furthermore, the rotation would separate the sample within the inner chamber 26′ to form at least another fraction of lower specific gravity, positioned as an annular concentric layer within the inner chamber 26 that is positioned closer to the longitudinal axis of the syringe assembly 10′, and/or closer to an air core at the central axis 6, than the outer layer of the first fraction. The first and second fractions, along with the air core are depicted in FIG. 12, Depicting the syringe assembly 10′ during longitudinal rotation about its axis, and the partial advancement of the container plunger 34′.

As before, in such an embodiment, the plunger assembly 14′ would provide dual plungers that are capable of independent operation, having previously been referred to as the PRP plunger 32, and the PPP plunger 34, and as depicted in FIGS. 5A, 5B, and 10, and now referred to as the ejection plunger 32′ and container plunger 34′. In the embodiment of the syringe assembly 10′, the container plunger 34′ would be advanced into the inner chamber 26′, and as it is advanced, a fraction would be caused to pass through the entrance port 38′, and be collected within the containment area 88′, and to thereby create a physical barrier separating the isolated fraction (positioned to the outside of the advanced container plunger 34′), and maintain separation from the other fraction (now positioned to the inside of the container plunger 34′ within the containment area 88′). If so desired, the ejection plunger 32′ may optionally be advanced to expel the other fraction from the syringe assembly 10′ for collection.

In the alternative embodiment, the syringe assembly 10′ may provide a plunger assembly 14′ having a container plunger 34′, corresponding in form and function to the previously described PPP separator plunger 34, and having a tapered leading edge with an entrance port 38′, for collecting the first fraction within the container 88′. The plunger assembly 14′ may also provide an ejection plunger 32′, corresponding in form and function to the previously described PRP plunger 32, and may optionally have an O-ring positioned at the distal end of the ejection plunger 32 to provide an improved seal at the distal end of the ejection plunger 32′ and aid in slidable advancement of the ejection plunger as needed. The syringe assembly 10′ may further provide a syringe body 12′ having an outer wall 20′ and a hollow interior defining inner chamber 26′. In the syringe assembly 10′, the RBC/PRP wall and slider drain actuator previously described with reference to the syringe body 12 of the syringe assembly 10, may be absent, as there would be no need to isolate a third fraction with the syringe assembly 10′.

With the heterogenous fluid sample introduced into the syringe assembly 10′, the syringe assembly 10′ may be spun as described previously about its longitudinal axis 6′ to separate the sample into fractions containing viable cells and non-viable cells, arranged as concentric annular layers within the syringe body 12′, and shown as first and second fractions in FIG. 12. The annular layers are formed by rotation of the sample within the chamber 26′, which urges the mixture outwards within the chamber, such that it can differentially flow under the centripetal force to form a density gradient, and eventually, provide first and second fractions as show, arranged about a central air core within the chamber 26′. The plunger assembly 14′ may then be actuated as previously described, for example, whether manually, or by use of a linear drive motor, while maintaining the rotation of the syringe assembly 10′ about the longitudinal axis, in order to advance the container plunger 34′, eventually causing the first fraction to be forced towards the port 38′ passing through valve 36′, thereby capturing the first fraction within the interior chamber 88′ of the container plunger 34′, as the first fraction is directed to pass from the interior of the syringe body 12′ and caused to enter into the interior chamber 88′, as the first fraction encounters the advancing conical entry of the container plunger 34′, whereby the first fraction is caused to pass through the flap valve 36′, thereby capturing the first fraction within the interior chamber 88′. It anticipated that such movement through the port 38′ would generally occur as the container plunger 34′ is advanced to the point that the first fraction is decreased in longitudinal length, and correspondingly increases in annular dimension, until the first fraction will be allowed to flow through the port 38′, and the container plunger 34 advances towards the conical protruding surface of the distal cap 42′. So long as the rotation about the longitudinal axis is maintained, the second fraction will remail positioned to the outside of the first fraction, as the outer concentric outer layer while the container plunger is advanced.

In the syringe assembly 10′, the plunger assembly 14′ will be dimensioned such that the container plunger 34′ has an outer diameter that would generally correspond to the interface between the first and second fraction (the interface defining the boundary between the viable cell portion and the non-viable cell portion) as the syringe assembly 10′ is caused to rotate. The container plunger 34′ provides an annular leading edge that will be aligned with the interface between the inner and outer fraction, and the conical entry is provided to funnel the second fraction, corresponding to the portion with the lower specific gravity, along the leading edges of the tapered conical design, and direct the fraction through the entrance port 38′ and flap valve 36 and into the interior chamber 88′.

The advancement of the container plunger 34′ may be performed as part of a timed sequence, initiated upon the start of the rotation of the separation syringe assembly 12′. For example, the centrifuge system may actuate a screw drive system, that advances the container plunger 34′ into the interior chamber 26′ of the syringe body 12′. The driving motion creates a pressure in the inner chamber 26′ and forces the second fraction through the flap valve 36 into the interior chamber 88′ within the container plunger 34′ until fully depressed (in contact with the end cap 42), in a position corresponding the that depicted in FIG. 5C, such that substantially all of the first fraction has been urged through the flap valve 36 into the interior chamber 88′, and leaving the second fraction, left in the space remaining between the exterior of the distally advanced container plunger 34′, and the interior surface of the outer wall 20′ of the syringe body 12′. After capturing substantially all of the first fraction within the interior chamber 88′, the mechanism for advancing the container plunger may be reversed, optionally leaving the container plunger 34′ in the advanced position, whereupon the rotation of the syringe assembly 12′ may be halted, and the first fraction may be accessed from the container 88′ or prepared for storage or delivery of the first fraction.

The separation syringe 10′ includes an access port for introducing a sample therein, such as a luer fitting or other fitting known in the art. The separation syringe 10′ is also provided with a luer fitting 44′ to which a needle, or another syringe or container may be mounted, for ejecting the isolated second fraction from the inner chamber, after the fractions have been isolated. The separation syringe 10′ may also provide a pierceable septum 46′, through which a syringe needle can be inserted to retrieve all or some of the isolated fraction from the interior of the container plunger 34′.

In an exemplary embodiment, the contents of the interior chamber 88′ may be accessed by directing a needle from a collection syringe through the distal end cap 42′, and through the flap valve 36′, to thereby allow the aspiration of the viable cell fraction. Alternatively, the container plunger 34′ may be removed from the syringe assembly 10′, and the contents of the interior chamber accessed or stored for eventual use as will be familiar to those of ordinary skill in the art.

Once the container plunger 34′ has been fully advanced in a distal direction, the second fraction will remail captured in the space between the outside of the container plunger 34′ and the inside of the outer wall 20′ of the syringe body 12′. To access the second fraction, if so desired, the ejection plunger 32′ may be advanced in a distal direction, where the ejection plunger 32 displaces the second fraction, causing it to be ejected through Luer fitting 44′ for collection, as has been previously described.

In an embodiment, the syringe assembly 10′ may further provide one or more support tabs 51′, positioned as protruding tabs, positioned at one or more points about the perimeter of the distal end of the container plunger 34′. These tabs serve to maintain alignment of the container plunger and the ejection plunger and to prevent unwanted movement or wiggle of the plunger assembly 14′ as the syringe assembly 10′ is rotated about its longitudinal axis. In an embodiment at least three support tabs 51 are positioned about the perimeter of the distal end of the container plunger 34′, such that oscillating movement is prevented, but leaving the rest of the perimeter of the distal end of the container plunger 34′ free from contact with the syringe barrel 12′, such as will minimize impact upon the interface between the first and second fraction as the container plunger 34′ is caused to advance.

The foregoing illustrates some of the possibilities for practicing the invention. Many other embodiments are possible within the scope and spirit of the invention. The disclosed invention utilizes the above identified components, as a system, in order to more efficiently separate a sample of biologic fluid into distinct isolated fractions for a particular purpose. Therefore, more, or less of the aforementioned components can be used to conform to that particular purpose. It is, therefore, intended that the foregoing description be regarded as illustrative, rather than limiting, and that the scope of the invention is given by the appended claims together with their full range of equivalents.

What is claimed is:

1. A separation syringe assembly comprising: a syringe barrel assembly and a plunger assembly configured to travel relative to the syringe barrel,
the syringe barrel assembly having a distal end and a proximal end with a longitudinal axis extending therebetween, the syringe barrel assembly having an outer wall in the form of a hollow cylinder, and a cap provided at the distal end of the syringe barrel assembly;
the plunger assembly having a proximal end and a distal end and aligned with the longitudinal axis, the plunger assembly providing a first plunger and a second plunger, the first plunger is a generally cylindrical hollow barrel having an entrance port positioned about the longitudinal axis and near the distal end of the first plunger and enclosed on the proximal end to form a container therein, the distal end of the first plunger is complementary in shape to a corresponding face of the cap of the syringe barrel assembly, the second plunger provides a cylindrical body with an open distal end and concentrically aligned outside of the first plunger;
wherein the separation syringe is configured to be rotated about the longitudinal axis and thereby separate the contents within the hollow cylinder of the syringe barrel assembly into at least two concentric annular layers.

2. The separation syringe assembly of claim 1, the entrance port of the first plunger has a one-way valve positioned adjacent to the entrance port.

3. The separation syringe assembly of claim 1, wherein the first plunger has an inverse conical face at the distal end of the first plunger, with the entrance port provided at the peak of the inverse conical face.

4. The separation syringe assembly of claim 3, wherein the cap is provided with a conical feature that is centrally aligned on an inside face of the cap, the conical feature capable of engaging with the inverse conical face of the first plunger.

5. The separation syringe assembly of claim 3, wherein the cap is further provided with a first access passage in fluid communication with the inner chamber, and offset from the longitudinal axis.

6. The separation syringe assembly of claim 5, wherein the first access passage features a luer fitting, and can be closed with a cap.

7. The separation syringe assembly of claim 5, wherein the cap is provided with a second access passage that is aligned with the longitudinal axis, the second access passage providing a pierceable septum.

8. The separation syringe assembly of claim 6, wherein the cap is provided with a counterbalance that is offset from the longitudinal axis in the same amount as the first access passage and in an opposite direction, the counterbalance having a weight that is the same as the luer fitting and cap.

9. The separation syringe assembly of claim 1, wherein the first plunger and second plunger are capable of independent axial translation along the longitudinal axis.

10. The separation syringe assembly of claim 1, wherein the first plunger further comprises at least one support element configured to stabilize the first plunger and limit the lateral movement of the first plunger relative to the syringe barrel.

11. The separation syringe assembly of claim 10, wherein the at least one support element is a plurality of stabilizing tabs positioned between the outer wall and the inner wall.
